(19) United States
(12) Patent Application Publication
(10) Pub. No.: US 2018/0374583 A1
(43) Pub. Date: Dec. 27, 2018

(54) NOMOGRAM AND SURVIVAL PREDICTIONS FOR PANCREATIC CANCER

(71) Applicant: Abraxis BioScience, LLC, Summit, NJ (US)

(72) Inventors: David GOLDSTEIN, Randwick (AU); Chee LEE, Camperdown (AU); Chrystal LOUIS, Somerville, MA (US); Brian LU, Summit, NJ (US); Anita N. SCHMID, Berkeley Heights, NJ (US); Markus RENSCHLER, Fort Lauderdale, FL (US)

(21) Appl. No.: 15/981,276

(22) Filed: May 16, 2018

Related U.S. Application Data
(60) Provisional application No. 62/507,132, filed on May 16, 2017.
(60) Provisional application No. 62/622,661, filed on Jan. 26, 2018.

(51) Int. Cl. G16H 50/50; G16H 50/30; G06G 1/14

(52) U.S. Cl. CPC G16H 50/50; G06G 1/14; G16H 50/30

(57) ABSTRACT

The present invention provides nomograms and methods or predicting survival probabilities for patients diagnosed with metastatic pancreatic cancer based upon patient characteristics such as neutrophil to lymphocyte ratio, albumin level, Karnofsky performance status, the sum of the longest diameter of target lesions, liver metastasis, previous Whipple procedure, treatment with nab-paclitaxel, and analgesic use. In some aspects, the nomograms or methods are implemented by a non-transitory computer-readable storage medium.

### CROSS REFERENCE TO RELATED APPLICATIONS

This application claims priority of U.S. Provisional Application No. 62/507,132, filed May 16, 2017, and U.S. Provisional Application No. 62/622,661, filed Jan. 26, 2018, the disclosures of which are herein incorporated by reference in their entirety.

### BACKGROUND OF THE INVENTION

A nomogram is a graphical instrument that represents a multivariate predictive model to illustrate the relative impact individual factors can have on predicting an outcome of interest. Touijer K, Scardino P T. Nomograms for staging, prognosis, and predicting treatment outcomes. Cancer 2009; 115: 3107-3111. One of the primary strengths of a nomogram is its ability to incorporate multiple patient factors to predict a patient's numerical probability for a specific event. Balachandran V P, Gonen M, Smith J J, DeMatteo R P. Nomograms in oncology: more than meets the eye. The lancet oncology 2015; 16: e173-e180. Nomograms are increasingly being used in various types of cancer, such as ovarian (Lee C, Simes R, Brown C et al. Prognostic nomogram to predict progression-free survival in patients with platinum-sensitive recurrent ovarian cancer. Br J Cancer 2011; 105: 1144-1150), breast (Delpech Y, Bashour S I, Lousquy R et al. Clinical nomogram to predict bone-only metastasis in patients with early breast carcinoma. Br J Cancer 2015; 113: 1003-1009), prostate (Niu X, Li J, Das S K et al. Developing a nomogram based on multiparametric magnetic resonance imaging for forecasting high-grade prostate cancer to reduce unnecessary biopsies within the prostate-specific antigen gray zone. BMC Medical Imaging 2017; 17: 11), and gastrointestinal (Zhang Z, Luo Q, Yin X et al. Nomograms to predict survival after colorectal cancer resection without preoperative therapy. BMC Cancer 2016; 16: 658), but none are currently available in metastatic pancreatic cancer.

Therefore, there is a need for an individualized predictive tool to accurately predict survival in metastatic pancreatic cancer, which is known to have a poor prognosis.

### BRIEF SUMMARY OF THE INVENTION

Provided herein are exemplary nomograms for determining a survival probability in an individual diagnosed with metastatic pancreatic cancer. In some embodiments, the nomogram comprises one or more factor scales comprising values for one or more factors. In some embodiments, the nomogram comprises a points scale comprising points values. In some embodiments, the nomogram comprises a total points scale comprising total points values. In some embodiments, the nomogram comprises a prediction scale. In some embodiments, the one or more factor scales are correlated with the points scale and the total points scale is correlated with the prediction scale. In some embodiments, in response to receiving values for one or more factors, values for one or more factors are correlated with the points scale to determine one or more points values, the one or more points values are combined to determine a total points value, and the total points value is correlated with the prediction scale to output a survival probability.

In some embodiments, the nomogram provided herein is able to distinguish between low, intermediate, and high risk groups.

In some embodiments, provided herein is a method to predict a survival probability of an individual comprising receiving values for one or more factors for the individual, determining separate points value for each of the one or more factors based upon one or more factor scales that are correlated with a points scale; combining each of the separate point values together to yield a total points value; and correlating the total points value with a prediction scale to predict the survival probability of the individual.

In some embodiments, provided herein is a computer-implemented method to predict a survival probability of an individual diagnosed with metastatic pancreatic cancer comprising: receiving one or more input values for one or more factors, wherein the one or more input values are associated with the individual; after receiving the one or more input values, determining, for each of the one or more factors, a respective points value based upon a points scale and a respective factor scale correlated with the points scale; aggregating the respective point values for the one or more factors to yield a total points value; correlating the total points value with a prediction scale to predict the survival probability of the individual; and providing one or more outputs based on the predicted survival probability of the individual.

In some embodiments of any of the above nomograms and methods, the one or more factors comprise neutrophil to lymphocyte ratio, albumin level, Karnofsky performance status, sum of the longest diameter of target lesions, presence of liver metastasis, and previous Whipple procedure. In some embodiments, the one or more factors comprise two or more factors selected from neutrophil to lymphocyte ratio, albumin level, Karnofsky performance status, sum of the longest diameter of target lesions, presence of liver metastasis, and previous Whipple procedure. In some embodiments, the one or more factors comprise three or more factors selected from neutrophil to lymphocyte ratio, albumin level, Karnofsky performance status, sum of the longest diameter of target lesions, presence of liver metastasis, and previous Whipple procedure. In some embodiments, the one or more factors comprise four or more factors selected from neutrophil to lymphocyte ratio, albumin level, Karnofsky performance status, sum of the longest diameter of target lesions, presence of liver metastasis, and previous Whipple procedure. In some embodiments, the one or more factors comprise five or more factors selected from neutrophil to lymphocyte ratio, albumin level, Karnofsky performance status, sum of the longest diameter of target lesions, presence of liver metastasis, and previous Whipple procedure. In some embodiments, the one or more factors comprise six or more factors selected from neutrophil to lymphocyte ratio, albumin level, Karnofsky performance status, sum of the longest diameter of target lesions, presence of liver metastasis, and previous Whipple procedure. In some embodiments, the one or more factors comprise neutrophil to lymphocyte ratio, albumin level, Karnofsky performance status, sum of the longest diameter of target lesions, presence of liver metastasis, and previous Whipple procedure. In some embodiments, the one or more factors comprise CA19-9 level, age, number of metastatic sites, number of lesions and presence of lung metastasis. In some of these embodiments, treatment with nab-paclitaxel is not a factor.

In some embodiments of any of the above nomograms and methods, the one or more factors comprise neutrophil to lymphocyte ratio, albumin level, Karnofsky performance status, sum of the longest diameter of target lesions, presence of liver metastasis, treatment with nab-paclitaxel, and analgesic use. In some embodiments, the one or more factors comprise two or more factors selected from neutrophil to lymphocyte ratio, albumin level, Karnofsky performance status, sum of the longest diameter of target lesions, presence of liver metastasis, treatment with nab-paclitaxel, and analgesic use. In some embodiments, the one or more factors comprise three or more factors selected from neutrophil to lymphocyte ratio, albumin level, Karnofsky performance status, sum of the longest diameter of target lesions, presence of liver metastasis, treatment with nab-paclitaxel, and analgesic use. In some embodiments, the one or more factors comprise four or more factors selected from neutrophil to lymphocyte ratio, albumin level, Karnofsky performance status, sum of the longest diameter of target lesions, presence of liver metastasis, treatment with nab-paclitaxel, and analgesic use. In some embodiments, the one or more factors comprise five or more factors selected from neutrophil to lymphocyte ratio, albumin level, Karnofsky performance status, sum of the longest diameter of target lesions, presence of liver metastasis, treatment with nab-paclitaxel, and analgesic use. In some embodiments, the one or more factors comprise six or more factors selected from neutrophil to lymphocyte ratio, albumin level, Karnofsky performance status, sum of the longest diameter of target lesions, presence of liver metastasis, treatment with nab-paclitaxel, and analgesic use. In some embodiments, the one or more factors comprise neutrophil to lymphocyte ratio, albumin level, Karnofsky performance status, sum of the longest diameter of target lesions, presence of liver metastasis, treatment with nab-paclitaxel, and analgesic use. In some embodiments, the one or more factors comprise CA19-9 level, age, number of metastatic sites, number of lesions and presence of lung metastasis.

Also provided herein is a method of using the nomogram of any of the above embodiments comprising determining one or more factors of any of the factors provided herein and providing a survival probability.

In some embodiments, provided herein is a computer-implemented method of generating a survival probability of an individual diagnosed with metastatic pancreatic cancer comprising receiving input data for an individual diagnosed with metastatic pancreatic cancer, the input data comprising data for one or more factors of a set of factors; processing the input data with a processing system to determine one or more numerical values; and applying a numerical model associated with a predetermined period of time to the one or more numerical values to determine a survival probability for the predetermined period of time, the numeric model including one or more factors and one or more associated first weighting factor, the one or more factor receiving a value of the one or more numerical value, and providing an output. In some embodiments, the factors that receive value of numerical measures determined from the input data comprise one or more numerical measures of one or more of neutrophil to lymphocyte ratio, albumin level, Karnofsky performance status, sum of the longest diameter of target lesions, liver metastasis, treatment with nab-paclitaxel and analgesic use.

Also provided herein is a non-transitory computer-readable storage medium for generating a survival probability for an individual diagnosed with metastatic pancreatic cancer, the computer-readable storage medium comprising computer executable instructions, which, when executed cause a processing system to execute steps comprising: receiving input data for an individual diagnosed with metastatic pancreatic cancer, the input data comprising data for one or more factors of a set of factors; processing the input data to determine one or more numerical measures; applying a numerical model associated with a predetermined period of time to the one or more numerical measure the numerical model including one or more factors and one or more associated first weighting factor, the one or more factors receiving a value of the one or more numerical values; and providing an output. In some of these embodiments, the factors that receive values of numerical measures determined from the input data comprise one or more numerical measures of one or more of neutrophil to lymphocyte ratio, albumin level, Karnofsky performance status, sum of longest diameter of target lesions, liver metastasis, treatment with nab-paclitaxel, and analgesic use. In some of these embodiments, the factors that receive values of numerical measures determined from the input data comprise one or more numerical measures of one or more of neutrophil to lymphocyte ratio, albumin level, Karnofsky performance status, sum of longest diameter of target lesions, liver metastasis, and previous Whipple procedure.

In some embodiments of any of the above embodiments, the individual is human.

In some embodiments of any of the above embodiments, the survival probability is calculated at 6 months. In some embodiments, the survival probability is calculated at 9 months. In some embodiments, the survival probability is calculated at 12 months.

### BRIEF DESCRIPTION OF THE DRAWINGS

FIG. 1 is a nomogram to predict the overall survival of chemotherapy-naive patients with metastatic pancreatic cancer receiving nab-paclitaxel plus gemcitabine or gemcitabine alone that includes treatment with nab-paclitaxel as a factor.

FIG. 2 is a nomogram to predict the overall survival of chemotherapy-naive patients with metastatic pancreatic cancer that does not include treatment with nab-paclitaxel as a factor.

FIG. 3 is a flowchart depicting steps of exemplary methods for generating a survival probability for a patient diagnosed with metastatic pancreatic cancer.

FIG. 4 is a flowchart depicting steps of an exemplary method of generating a survival probability for a patient diagnosed with metastatic pancreatic cancer.

FIGS. 5A-5C depict exemplary systems for implementing the techniques described herein.

FIGS. 6A-6E show screenshots of exemplary user interfaces utilizing the systems and methods provided herein according to embodiments of the present invention.

FIG. 7 is a STROBE (Strengthening the Reporting of Observational Studies in Epidemiology) diagram of patient inclusion from the MPACT clinical trial.

FIGS. 8A-8C are calibration plots for 6-, 9-, and 12-month survival adjusted by bootstrapping (FIG. 8A) 6 months; (FIG. 8B) 9 months; (FIG. 8C) 12 months for a nomogram that includes treatment with nab-paclitaxel as a factor.

FIG. 9 shows Kaplan-Meier survival curves according to nomogram-predicted survival probabilities of low-, intermediate-, and high-risk patients for a nomogram that includes treatment with nab-paclitaxel as a factor.

FIGS. 10A-10C are calibration plots for 6-, 9-, and 12-month survival adjusted by bootstrapping (FIG. 10A) 6 months; (FIG. 10B) 9 months; (FIG. 10C) 12 months for a nomogram that does not include treatment with nab-paclitaxel as a factor.

FIG. 11 shows Kaplan-Meier survival curves according to nomogram-predicted survival probabilities of low-, intermediate-, and high-risk patients for a nomogram that does not include treatment with nab-paclitaxel as a factor.

### DETAILED DESCRIPTION OF THE INVENTION

Definitions

“Nab-paclitaxel” or “nab-P” as used herein is a nanoparticle composition comprising paclitaxel and albumin. In some embodiments nab-paclitaxel is Abraxane™ which is also sometimes called ABI-007.

“CA-19-9” as used herein is the tumor marker carbohydrate antigen 19-9.

“Gem” as used herein is gemcitabine including (Gemzar®).

“KPS” as used herein is Karnofsky performance status. KPS is based upon a 0-100 scale with an individual with no complaints (normally functioning) receiving a score of 100 and a dead individual receiving a score of 0. A KPS score of 90 indicates that an individual is able to carry on normal activity and has minor signs or symptoms of disease; a score of 80 indicates that the individual is able to carry on normal activity with effort and has some signs of disease; a score of 70 indicates that the individual cares for herself and is unable to carry on normal activity or do active work; a score of 60 indicates that the individual requires occasional assistance but is able to care for most of her personal needs; a score of 50 indicates that the individual requires considerable assistance and frequent medical care; a score of 40 indicates that the individual is disabled and require special care and assistance; a score of 30 indicates that the individual is severely disabled and hospital admission is indicated although death is not imminent; a score of 20 indicates that the individual is very sick and that hospital admission is necessary; and a score of 10 indicates that the individual is moribund and that the fatal processes are progressing rapidly.

“NLR” as used herein is neutrophil to lymphocyte ratio. NLR is calculated by dividing the number of neutrophils by the number of lymphocytes, usually from peripheral blood samples. In some embodiments, NLR can also be calculated from cells that infiltrate tissues such as tumors.

“OS” as used herein is overall survival.

“SLD” as used herein is the sum of the longest tumor diameters. The sum of the longest diameter of target lesions can be obtained from radiographic scans. CT and MRI can be used to measure target lesions. Conventional CT and MRI can be performed with cuts of 10 mm or less in slice thickness contiguously. Spiral CT can be performed using a 5 mm contiguous reconstruction algorithm. In some embodiments the sum of the longest diameter of target lesions is determined using Response Evaluation Criteria for Solid Tumors (RECIST) criteria. In some of these embodiments, a maximum 5 target organs are considered and a maximum of 10 lesions total. The longest diameter of a target lesion can be measured in centimeters.

The term “individual” as used herein is a human. In some embodiments, the individual has metastatic pancreatic cancer.

The term “palliative” or “palliation” refers to a type of care or treatment that is focused on providing relief from the symptoms and stress of a serious illness. The goal is to improve quality of life for both the patient and the family.

The methods may be practiced in an adjuvant setting. “Adjuvant setting” refers to a clinical setting in which an individual has had a history of a proliferative disease, particularly cancer, and generally (but not necessarily) been responsive to therapy, which includes, but is not limited to, surgery (such as surgical resection), radiotherapy, and chemotherapy. However, because of their history of the proliferative disease (such as cancer), these individuals are considered at risk of development of the disease. Treatment or administration in the “adjuvant setting” refers to a subsequent mode of treatment. The degree of risk (i.e., when an individual in the adjuvant setting is considered as “high risk” or “low risk”) depends upon several factors, most usually the extent of disease when first treated. The methods provided herein may also be practiced in a neoadjuvant setting, i.e., the method may be carried out before the primary/definitive therapy. In some embodiments, the individual has previously been treated. In some embodiments, the individual has not previously been treated. In some embodiments, the treatment is a first line therapy.

The term “effective amount” used herein refers to an amount of a compound or composition sufficient to treat a specified disorder, condition or disease such as ameliorate, palliate, lessen, and/or delay one or more of its symptoms. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay occurrence and/or recurrence. An effective amount can be administered in one or more administrations. In the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

Nomograms

Nomograms are prediction tools that can be used to help patients and their physicians understand the nature of their cancer, assess risk based upon specific characteristics of a patients and his disease, and predict the likely outcomes of treatment, such as the survival probability of the patient at a particular time. Nomograms can also be used to aid patients and physicians in selecting a course of treatment based upon a patient's survival probability. Relevant characteristics or “factors” for the present nomogram which can be used to predict survival probability in an individual diagnosed with metastatic pancreatic cancer include those described herein such as neutrophil to lymphocyte ratio, albumin level, Karnofsky performance status, sum of the longest diameter of target lesion, presence of liver metastasis, treatment with nab-paclitaxel, previous Whipple procedure, or analgesic use. Non-invasive assays are also provided by the invention to detect and/or quantitate neutrophil to lymphocyte ratio, albumin level, Karnofsky performance status, sum of the longest diameter of target lesion, presence of liver metastasis, treatment with nab-paclitaxel, or analgesic use.

TABLE 1
Exemplary scoring system for metastatic pancreatic cancer nomogram
including treatment with nab-paclitaxel as a factor.

| Factor | Points |
|---|---|
| Neutrophil-to-lymphocyte ratio | |
| 80 | 100 |
| 60 | 75 |
| 40 | 50 |
| 20 | 25 |
| 0 | 0 |
| Albumin level (g/L) | |
| 10 | 80 |
| 20 | 64 |
| 30 | 48 |
| 40 | 32 |
| 50 | 16 |
| 60 | 0 |
| Karnofsky performance status | |
| 60 | 28 |
| 70 | 21 |
| 80 | 14 |
| 90 | 7 |
| 100 | 0 |
| Sum of the longest diameter of target lesions (cm) | |
| 50 | 19 |
| 40 | 15 |
| 30 | 11 |
| 20 | 8 |
| 10 | 4 |
| 0 | 0 |
| Presence of liver metastases | |
| Yes | 12 |
| No | 0 |
| Treatment arm | |
| Gemcitabine alone | 11 |
| nab-Paclitaxel plus gemcitabine | 0 |
| Analgesic use | |
| Yes | 4 |
| No | 0 |

TABLE 2
Exemplary scoring system for metastatic pancreatic cancer nomogram
excluding treatment with nab-paclitaxel as a factor

| Factor | Points |
|---|---|
| Neutrophil-to-lymphocyte ratio | |
| 80 | 100 |
| 60 | 75 |
| 40 | 50 |
| 20 | 25 |
| 0 | 0 |
| Albumin level (g/L) | |
| 0 | 86 |
| 10 | 72 |
| 20 | 57 |
| 30 | 43 |
| 40 | 29 |
| 50 | 14 |
| 60 | 0 |
| Karnofsky performance status | |
| 60 | 23 |
| 70 | 18 |
| 80 | 12 |
| 90 | 6 |
| 100 | 0 |
| Sum of the longest diameter of target lesions (cm) | |
| 0 | 0 |
| 10 | 4 |
| 20 | 7 |
| 30 | 11 |
| 40 | 15 |
| 50 | 18 |
| Presence of liver metastases | |
| Yes | 9 |
| No | 0 |
| Previous Whipple procedure | |
| Yes | 0 |
| No | 6 |

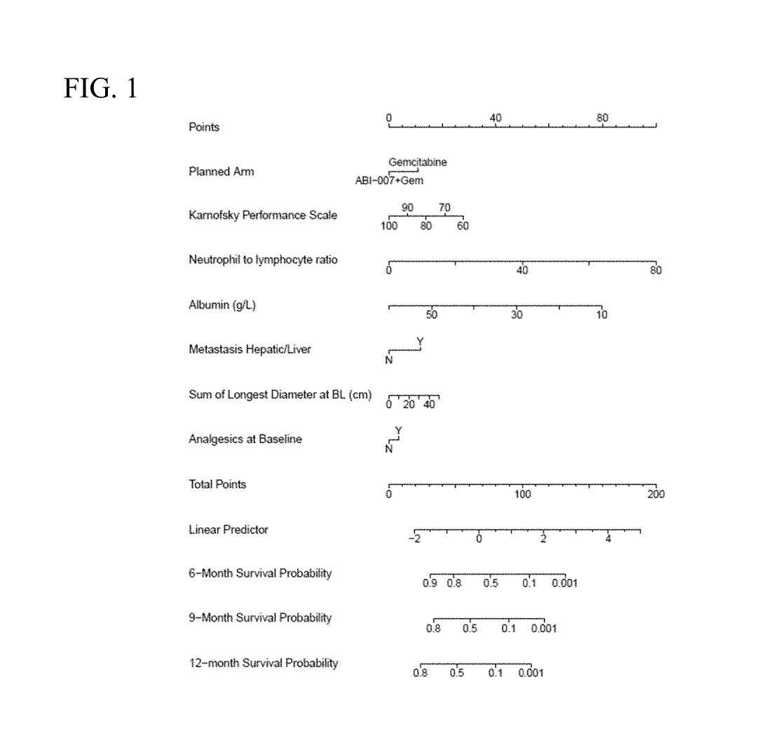

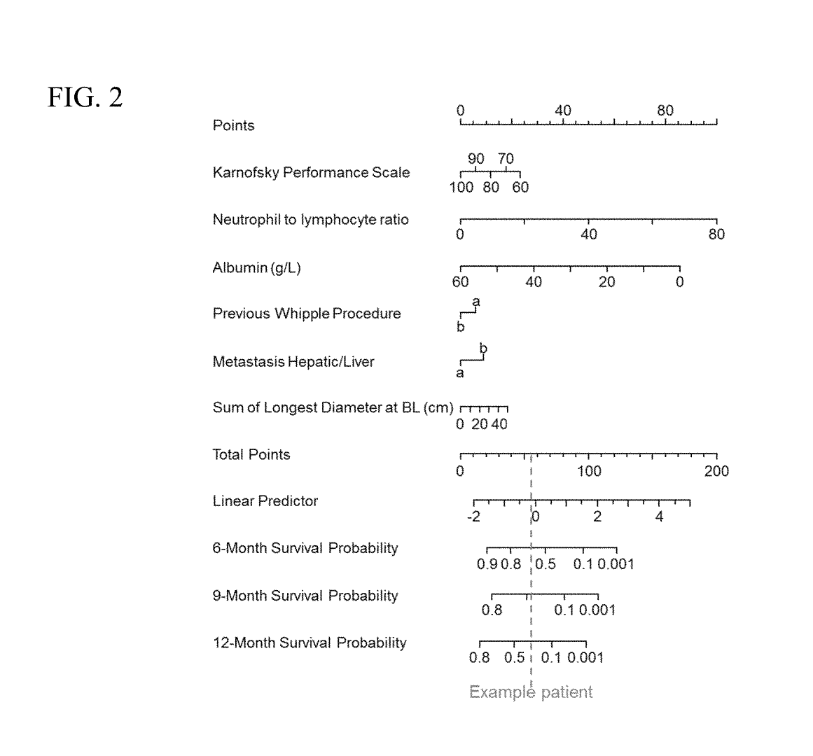

In some embodiments, neutrophil to lymphocyte ratio (NLR) is a factor used in the nomogram provided herein. NLR is calculated by dividing the number of neutrophils by the number of lymphocytes, usually from peripheral blood samples. In some embodiments, NLR can also be calculated from cells that infiltrate tissues such as tumors. In the present nomogram, a higher NLR is correlated with a higher points value, which is correlated to a lower survival probability. In some embodiments the present nomogram contains a factor scale for NLR that ranges from a value of 0 to 80. In some embodiments, the NLR factor scale is correlated with the points scale as shown in FIG. 1 or FIG. 2. In some embodiments, the NLR value is correlated with points values as shown in Table 1 or Table 2. For example a NLR of 80 correlates to 100 points, a NLR of 60 correlates to 75 points, a NLR of 40 correlates to 50 points, a NLR of 20 correlates with 25 points, and a NLR of 0 correlates with 0 points. In some embodiments the neutrophil to lymphocyte ratio is the most heavily weighted factor in the nomogram.

Albumin is a protein that is made by the liver. In some embodiments, albumin level is a factor that is used in the nomogram provided herein. The presence and amount of albumin can be detected in the blood, serum, or urine of an individual. In some embodiments albumin level is measured as grams per liter of blood. In the nomogram provided herein a lower albumin level is correlated with a higher points value which is correlated with a lower survival probability. In some embodiments, the present nomogram contains a factor scale for albumin level that ranges from a value of 10 g/L to a value of 60 g/L. In some embodiments, the albumin factor scale is correlated to the points scale as shown in FIG. 1. In some embodiments, the albumin level is correlated to the points values as shown in Table 1. In some embodiments, an albumin level of 10 g/L correlates with 80 points, an albumin level of 20 g/L correlates with 64 points, an albumin level of 30 g/L correlates to 48 points, an albumin level of 40 g/L correlates to 32 points, an albumin level of 50 g/L correlates to 16 points, and an albumin level of 60 g/L correlates to 0 points. In some embodiments the albumin level is the second most heavily weighted factor in the nomogram.

In some embodiments, when treatment with nab-paclitaxel is not included in the nomogram, the albumin level is correlated to the points values as shown in Table 2. In some embodiments, the albumin level is correlated to the points values as shown in Table 1. IN some embodiments, a higher albumin level correlates to a lower points value. In some embodiments, an albumin level of 0 g/L correlates with 86 points. In some embodiments, an albumin level 10 g/L correlates with 72 points, an albumin level of 20 g/L correlates with 57 points, an albumin level of 30 g/L correlates to 43 points, an albumin level of 40 g/L correlates to 29 points, an albumin level of 50 g/L correlates to 14 points, and an albumin level of 60 g/L correlates to 0 points.

Karnofsky performance status (or KPS) allows classification of patients according to their functional impairment. In some embodiments, KPS is a factor in the present nomogram. KPS is based upon a 0-100 scale with a normal individual with no complaints and no evidence of disease receiving a score of 100 and a dead individual receiving a score of 0. In some embodiments, a KPS score of 90 indicates that an individual is able to carry on normal activity and has minor signs or symptoms of disease; a score of 80 indicates that the individual is able to carry on normal activity with effort and has some signs of disease; a score of 70 indicates that the individual cares for herself and is unable to carry on normal activity or do active work; a score of 60 indicates that the individual requires occasional assistance but is able to care for most of her personal needs; a score of 50 indicates that the individual requires considerable assistance and frequent medical care; a score of 40 indicates that the individual is disable and require special care and assistance; a score of 30 indicates that the individual is severely disabled and hospital admission is indicated although death is not imminent; a score of 20 indicates that the individual is very sick and that hospital admission is necessary; and a score of 10 indicates that the individual is moribund and that the fatal processes are progressing rapidly. In some embodiments, the KPS status of an individual is determined by a doctor, such as an oncologist. In some embodiments, the KPS status of an individual is determined by a healthcare professional who is not a doctor. In some embodiments the present nomogram contains a factor scale for KPS that ranges from 60 to 100. In some embodiments, the KPS factor scale is correlated with the points scale such that a lower KPS is correlated with a higher points value, which is correlated with a lower survival probability.

In some embodiments the KPS factor scale is correlated with the points scale as shown in FIG. 1. In some embodiments, KPS values are correlated with points values as shown in Table 1. In some embodiments, a KPS value of 60 correlates to a points value of 28, a KPS value of 70 correlates with a points value of 21, a KPS value of 80 correlates with a points value of 14, a KPS value of 90 correlates with a points value of 7, and a KPS value of 100 correlates with a points value of 0. In some embodiments the KPS score is the third most heavily weighted factor in the nomogram.

In some embodiments when treatment with nab-paclitaxel is not included as a factor, the KPS factor scale is correlated with the points scale as shown in FIG. 2. In some embodiments, KPS values are correlated with points values as shown in Table 2. In some embodiments, a KPS value of 60 correlates to a points value of 23, a KPS value of 70 correlates with a points value of 18, a KPS value of 80 correlates with a points value of 12, a KPS value of 90 correlates with a points value of 6, and a KPS value of 100 correlates with a points value of 0. In some embodiments the KPS score is the third most heavily weighted factor in the nomogram.

In some embodiments, the nomogram provided herein comprises the factor of sum of the longest diameter of target lesions (SLD). In some embodiments, the sum of the longest diameter of target lesions can be obtained from radiographic scans. CT and MRI can be used to measure target lesions. Conventional CT and MRI can be performed with cuts of 10 mm or less in slice thickness contiguously. Spiral CT can be performed using a 5 mm contiguous reconstruction algorithm. In some embodiments the sum of the longest diameter of target lesions is determined using Response Evaluation Criteria for Solid Tumors (RECIST) criteria. In some of these embodiments, a maximum 5 target organs are considered and a maximum of 10 lesions total that are representative of the patient's overall disease. In some embodiments the longest diameter of a target lesion is measured in centimeters. In the nomogram provided herein a higher sum of the longest diameter of target lesions is correlated with a higher points value which is correlated with a lower survival probability. In some embodiments the present nomogram contains a factor scale for the sum of the longest diameter of target lesions that ranges from a value of 0 cm to 50 cm.

In some embodiments, the factor scale of the sum of the longest diameter of target lesions is correlated with the points scale as shown in FIG. 1. In some embodiments, a value of the sum of the longest diameter of target lesions corresponds to a points value as shown in Table 1. In some embodiments, a SLD of 50 correlates with 19 points, a SLD of 40 correlates to 15 points, a SLD of 30 correlates to 11 points, a SLD of 20 correlates to 8 points, a SLD of 10 correlates to 4 points, and a SLD of 0 correlates to 0 points. In some embodiments the sum of the longest diameter of target lesions is the fourth most heavily weighted factor in the nomogram.

In some embodiments, when treatment with nab-paclitaxel is not included as a factor, the factor scale of the sum of the longest diameter of target lesions is correlated with the points scale as shown in FIG. 2. In some embodiments, a value of the sum of the longest diameter of target lesions corresponds to a points value as shown in Table 2. In some embodiments, a SLD of 50 correlates with 18 points, a SLD of 40 correlates to 15 points, a SLD of 30 correlates to 11 points, a SLD of 20 correlates to 7 points, a SLD of 10 correlates to 4 points, and a SLD of 0 correlates to 0 points.

In some embodiments, the present nomogram also comprises the factor of the presence of liver metastasis. In the nomogram provided herein the presence of liver metastasis is correlated with a higher points value which is correlated with a lower survival probability. In some embodiments, the present nomogram contains a factor scale for the presence of liver metastasis which comprises two points: yes and no. In some embodiments, the factor scale for the presence of liver metastasis is correlated with the points scale such that the presence of liver metastasis is correlated with a higher points value. In some embodiments, the factor scale for the presence of liver metastasis is correlated with the points scale as shown in FIG. 1. In some embodiments, the presence of liver metastasis correlates with 12 points and the absence of liver metastasis correlates with 0 points as shown in Table 1. In some embodiments the sum of the presence of liver metastasis is the fifth most heavily weighted factor in the nomogram.

In some embodiments, when treatment with nab-paclitaxel is not included as a factor in the nomogram, the factor scale for the presence of liver metastasis is correlated with the points scale such that the presence of liver metastasis is correlated with a higher points value. In some embodiments, the factor scale for the presence of liver metastasis is correlated with the points scale as shown in FIG. 2. In some embodiments, the presence of liver metastasis correlates with 9 points and the absence of liver metastasis correlates with 0 points as shown in Table 2.

In some embodiments, the present nomogram comprises the factor of whether the individual has been treated with nab-paclitaxel. In some embodiments of the present nomogram treatment with nab-paclitaxel is correlated with a lower points value which is correlated with a higher survival probability. In some embodiments the present nomogram contains a factor scale for the treatment with nab-paclitaxel which comprises two points: yes and no. In some embodiments, the factor scale of treatment with nab-paclitaxel is correlated with the points scale as shown in FIG. 1. In some embodiments, treatment with nab-paclitaxel correlates with 0 points and no treatment with nab-paclitaxel correlates to 11 points, as shown in Table 1. In some embodiments treatment with nab-paclitaxel is the sixth most heavily weighted factor in the nomogram.

In some embodiments, the present nomogram does not comprise the factor of whether the individual has been treated with nab-paclitaxel. In some of these embodiments, the nomogram comprises the factor of whether the subject has previous had a Whipple procedure. A Whipple procedure, also known as a pancreaticoduodenectomy, can involve removal of the “head” or wide part of the pancreas next to the duodenum. It also involves removal of the duodenum, a portion of the common bile duct, gallbladder, and sometimes part of the some stomach. In some embodiments, having a Whipple procedure is correlated with a lower points value, which is correlated with a higher survival probability. In some embodiments the present nomogram contains a factor scale for previous Whipple procedure which comprises two points: yes and no. In some embodiments, the factor scale of previous Whipple procedure is correlated with the points scale as shown in FIG. 2. In some embodiments, previous Whipple procedure correlates with 0 points and no previous Whipple procedure correlates with 6 points. In some embodiments, previous Whipple procedure is the sixth most heavily weighted factor in the nomogram.

In some embodiments, the present nomogram comprises the factor of whether the patient is using analgesics. An analgesic or painkiller is any member of the group of drugs used to achieve analgesia, relief from pain. Classes of analgesics include NSAIDS, COX-2 inhibitors, opioids, and medical cannabis. In some embodiments of the present nomogram use of analgesics is correlated with a higher points value which is correlated with a lower survival probability. In some embodiments the present nomogram comprises a factor scale for the use of analgesics which comprises two points: yes and no. In some embodiments, the factor scale for the use of analgesics is correlated with the points scale such that use of analgesics is correlated with a higher points value. In some embodiments, the analgesic use factor scale is correlated with the points scale as shown in FIG. 1. In some embodiments, use of analgesic is correlated with 4 points and non-use of analgesics is correlated with 0 points as shown in table 1. In some embodiments the use of analgesics is the seventh most heavily weighted factor in the nomogram. In some embodiments, the factor of whether the patient is using analgesics is used in a nomogram that includes treatment with nab-paclitaxel as a factor.

In some embodiments, the nomogram does not comprise previous use of analgesics as a factor.

In some embodiments, the nomogram provided herein comprises factor scales for 1, 2, 3, 4, 5, 6, or all 8 of factors described above. For example, in some embodiments, the nomogram comprises the factors NLR, albumin level, KPS, sum of the longest diameter of target lesions, presence of liver metastasis, treatment with nab-paclitaxel, and use of analgesics. In some embodiments, the nomogram comprises the factors NLR, albumin level, KPS, sum of the longest diameter of target lesions, presence of liver metastasis, and treatment with nab-paclitaxel. In some embodiments, the nomogram comprises the factors NLR, albumin level, KPS, sum of the longest diameter of target lesions, and presence of liver metastasis. In some embodiments, the nomogram comprises the factors NLR, albumin level, KPS, and of the longest diameter of target lesions. In some embodiments, the nomogram comprises the factors NLR, albumin level and KPS. In some embodiments, the nomogram comprises the factors NLR and albumin level.

In some embodiments, the nomogram comprises the factors NLR, albumin level, KPS, sum of the longest diameter of target lesions, presence of liver metastasis, and use of analgesics. In some embodiments, the nomogram comprises the factors NLR, albumin level, KPS, presence of liver metastasis, and use of analgesics. In some embodiments, the nomogram comprises the factors NLR, albumin level, KPS, presence of liver metastasis, and use of analgesics. In some embodiments, the nomogram comprises the factors NLR, albumin level, sum of the longest diameter of target lesions, presence of liver metastasis, and use of analgesics. In some embodiments, the nomogram comprises the factors NLR, KPS, sum of the longest diameter of target lesions, presence of liver metastasis, and use of analgesics.

In some embodiments, the nomogram comprises the factors NLR, albumin level, KPS, sum of the longest diameter of target lesions, presence of liver metastasis. In some embodiments, the nomogram comprises the factors NLR, albumin level, KPS, sum of the longest diameter of target lesions, and presence of liver metastasis. In some embodiments, the nomogram comprises the factors NLR, albumin level, KPS, presence of liver metastasis and previous Whipple procedure. In some embodiments, the nomogram comprises the factors NLR, albumin level, sum of the longest diameter of target lesions, presence of liver metastasis, and previous Whipple procedure. In some embodiments, the nomogram comprises the factors NLR, KPS, sum of the longest diameter of target lesions, presence of liver metastasis, and previous Whipple procedure.

In some embodiments the nomogram provided herein comprises additional factors such as CA19-9 level; number of metastatic sites; number of lesions; presence of lung metastasis; age; gender; race/ethnicity; height; weight; body mass index; body surface area; presence of a biliary stent; location of primary tumor in the pancreases (head, body, or tail); presence of metastasis in the abdomen/perioteneum, axilla, bone, breast, groin, hepatic, lung, thoracic, pelvis, periotoneal carcinmatosis, skin/soft tissue, and supraclavicular; number of metastatic sites; previous whipple procedure; prior chemotherapy; and prior radiation.

CA19-9 (Cancer Antigen 19-9) is a tumor marker that has been used in some instances for the detection and/or prognosis of pancreatic cancer. In some embodiments, the present nomogram does not comprise a factor scale for CA19-9.

In the present nomogram, each of one or more factor scales is correlated with a points scale such that a value on a factor scale is correlated with a points value. In some embodiments, the points scale ranges from 0 to 100. The points values for each factor are combined, for example by adding each of the points values, to calculate a total points value. In some embodiments, the present nomogram comprises a total points scale that ranges from 0 to 200.

In some embodiments, the total points scale is correlated with one or more prediction scales. In some embodiments, each prediction scale corresponds to the likelihood of survival of an individual at a particular time point. For example, in some embodiments, the present nomogram comprises prediction scales for survival at 6, 9, and 12 months. In some embodiments, the present nomogram comprises prediction scales for survival at 1, 2, 3, 4, 5, 6, 7, 8, 9, 10, 11, 12, 13, 14, 15, 16, 17 18, 19, 20, or 24 months. In some embodiments, the one or more prediction scales range from 0.9 to 0.001, where a value of 0.9 on the prediction scale indicates that an individual has a 90% likelihood of survival at a particular time point, for example at 6 months.

In some embodiments, the present nomogram is especially suitable for predicting survival probability in individuals who have received gemcitabine or gemcitabine plus nab-paclitaxel. In some embodiments the individual has not received prior chemotherapy in the adjuvant or metastatic setting before treatment with gemcitabine or gemcitabine plus nab-paclitaxel. In some embodiments, the individual has received prior radiation therapy. In some embodiments, the individual has received 5-fluoruracil and/or gemcitabine as a sensitizer prior to radiation therapy. In some embodiments, the present nomogram is suitable for predicting survival probability in individuals with metastatic pancreatic cancer, independent of whether the individual has received gemcitabine or gemcitabine plus nab-paclitaxel.

In some embodiments, the present nomogram is used to predict the survival probability of an individual diagnosed with pancreatic cancer. In some embodiments, the present nomogram is used to predict the survival probability of an individual diagnosed with advanced pancreatic cancer. In some embodiments, the present nomogram is used to predict survival probability of a patient diagnosed with metastatic pancreatic cancer. In some embodiments, the present nomogram is used to predict survival probability of an individual diagnosed with stage IVA pancreatic cancer. In some embodiments, the individual has metastatic adenocarcinoma of the pancreas.

In some embodiments, the present nomogram is used to predict the survival probability of an individual who has a KPS of greater than or equal to 70. In some embodiments, the present nomogram is used to predict survival probability of an individual who has a bilirubin level less than or equal to the upper limit of normal.

Methods of Predicting a Survival Probability

Also provided herein are methods for predicting a survival probability of an individual diagnosed with metastatic pancreatic cancer. In some embodiments, the method of predicting a survival probability in an individual comprises receiving values for one or more factors for an individual; determining a separate points value for each of the one or more factors based upon one or more factor scales, that are correlated with a points scale, combining each of the separate point values together to yield a total points value and correlating the total points value with a prediction scale to predict the survival probability of the individual.

In some embodiments, the one or more factors comprise any of the factors described herein, for example NLR, albumin level, KPS, sum of the longest diameter of target lesions, presence of liver metastasis, treatment with nab-paclitaxel, and use of analgesics. In some embodiments, the method comprises receiving values for 2 or more, 3, or more, 4, or more, 5, or more, 6 or more, or 7 or factors comprising NLR, albumin level, KPS, sum of the longest diameter of target lesions, presence of liver metastasis, treatment with nab-paclitaxel, and use of analgesics. In some embodiments, the method comprises receiving values for 2 or more, 3, or more, 4, or more, 5, or more, 6 or more, or 7 or factors comprising NLR, albumin level, KPS, sum of the longest diameter of target lesions, presence of liver metastasis, and previous Whipple procedure.

In some embodiments, the survival probability can be predicted at any given time point based upon the values for the one or more factors. Survival probability is the likelihood that a patient will be alive at a particular time or a range of time. For example, a survival probability of 0.9 at 6 months indicates that based upon the values of the factors, the individual has a 90% likelihood of being alive at 6 months. Survival probability of an individual can be calculated for any of 1, 2, 3, 4, 5, 6, 7, 8, 9, 10, 11, 12, 13, 14, 15, 16, 17, 18, 19, 20, or 24 months. Survival probability can also be calculated for any range of time. In some embodiments, survival probably can be calculated at 3 to 6 months, 4 to 6 months, 6 to 9 months 6 to 12 months, 9 to 12 months, etc.

In some embodiments, the present methods can also be used to calculate the probability that the individual may die at a given time or at a range of times. For example, a 0.1 probability of death at 6 months indicates that based upon the values of the factors, the individual has a 10% likelihood of dying within 6 months. The probability that an individual will die an individual can be calculated for any of 1, 2, 3, 4, 5, 6, 7, 8, 9, 10, 11, 12, 13, 14, 15, 16, 17, 18, 19, 20, or 24 months. The probability that an individual will die probability can also be calculated for any range of time. In some embodiments the probability that an individual will die can be calculated at 3 to 6 months, 4 to 6 months, 6 to 9 months 6 to 12 months, 9 to 12 months, etc.

In some embodiments, the method of predicting a survival probability in an individual comprises receiving values for one or more factors for an individual; determining a separate points value for each of the one or more factors based upon one or more factor scales, that are correlated with a points scale, combining each of the separate point values together to yield a total points value and correlating the total points value with a prediction scale to predict the survival probability of the individual, and treating the individual based upon the survival probability of the individual. In some embodiments, the method of predicting a survival probability in an individual comprises receiving values for one or more factors for an individual; determining a separate points value for each of the one or more factors based upon one or more factor scales, that are correlated with a points scale, combining each of the separate point values together to yield a total points value and correlating the total points value with a prediction scale to predict the survival probability of the individual, and providing a treatment recommendation for the individual based upon the survival probability of the individual.

In some embodiments, a lower survival probability results in a more aggressive treatment recommendation to the individual. In some embodiments, a lower survival probability results in less aggressive treatment recommendation to the individual. In some embodiments, a lower survival probability results in a recommendation of palliative treatment to the individual. In some embodiments, the treatment recommendation comprising a recommendation other than treatment with gemcitabine and/or nab-paclitaxel.

In some embodiments, also provided herein is a method of patient stratification using the nomograms provided herein. For example, the nomograms provided herein can be used to calculate a survival probability that can be used to stratify patients into different groups (i.e., low, medium, and high risk of mortality) for clinical trials.

Methods of Treatment

Also provided herein are methods of treating a patient diagnosed with pancreatic cancer based upon a survival probability. In some embodiments, provided herein is method of treatment comprising determining a survival probability of a patient as described herein and providing a treatment recommendation. In some embodiments, the treatment recommendation is for a therapy other than gemcitabine and/or nab-paclitaxel.

In some embodiments, provided herein is a method of treatment comprising administering a first therapy comprising gemcitabine; receiving values for one or more factors for an individual; determining a separate points value for each of the one or more factors based upon one or more factor scales, that are correlated with a points scale; combining each of the separate point values together to yield a total points value and correlating the total points value with a prediction scale to predict the survival probability of the individual, and administering a second therapy based upon the survival probability of the individual. In some embodiments, the first therapy further comprises nab-paclitaxel In some embodiments, the first therapy is a first line therapy and the second therapy is a second line therapy. In some embodiments, the second therapy is chemotherapy. In some embodiments, the second therapy is capecitabine. In some embodiments, the second therapy is fluorouracil, leucovorin and oxaliplatin (FOLFOX). In some embodiments, the second therapy is oxaliplatin, irinotecan, fluorouracil, and leucovorin (FOLFIRINOX). In some embodiments, the second therapy is radiation therapy.

In some embodiments, the individual is treated with either nab-paclitaxel and gemcitabine or only gemcitabine prior to determining a survival probability. Eexemplary dosing schedules for the administration of the nab-paclitaxel composition (for example Abraxane®™) include, but are not limited to, 100 mg/m², weekly, without break; 75 mg/m² weekly, 3 out of four weeks; 100 mg/m², weekly, 3 out of 4 weeks; 125 mg/m², weekly, 3 out of 4 weeks; 125 mg/m², weekly, 2 out of 3 weeks; 130 mg/m², weekly, without break; 175 mg/m², once every 2 weeks; 260 mg/m², once every 2 weeks; 260 mg/m², once every 3 weeks; 180-300 mg/m², every three weeks; 60-175 mg/m², weekly, without break. In addition, the taxane (alone or in combination therapy) can be administered by following a metronomic dosing regime described herein. In some embodiments, the individual is administered 125 mg/m² of nab-paclitaxel followed by gemcitabine (1000 mg/m²) on days 1, 8, and 15 every 4 weeks. In some embodiments, the individual is administered gemcitabine (1000 mg/m²) weekly for 7 of 8 weeks (cycle 1) and then on days 1, 8, and 15.

The methods and nomograms provided herein are also useful to identify patients who may be suitable for a clinical trial, or for classifying patients within a clinical trial. The present methods and nomograms are useful for identify patient sub-populations with any given survival probability. For instance, in some embodiments, using the present nomograms and methods, a sub-population of patients having metastatic pancreatic cancer with a greater than 50% survival probability at 6 months can be identified. Likewise, the using the present nomograms and methods a sub-population of patients with a less than 25% survival probability at 9 months can be identified.

Computer-Implemented Methods

In some embodiments, provided herein is a computer-implemented method of generating a survival probability for an individual diagnosed with metastatic pancreatic cancer, the method comprising: receiving input data for an individual diagnosed with metastatic pancreatic cancer, the input data comprising data for one or more factors of a set of factors; processing the input data with a processing system to determine one or more numerical values; and applying a numerical model associated with a predetermined period of time to the one or more numerical values to determine a survival probability for the predetermined period of time, the numerical model including one or more factors and one or more associated first weighting factor, the one or more factors receiving a value of the one or more numerical value.

In some embodiments, the numerical model is a COX model. In some embodiments, the factors comprise values for one or more factors as described herein (for example, albumin level, NLR, analgesic use, etc.). In some embodiments, the model comprises factors chosen because of clinical relevance and/or their close proximity to the prespecified alpha level. In some embodiments, the model comprises factors that were identified as associated with overall survival in a statistically significant manner in a multivariate model.

Also provided herein is a non-transitory computer-readable storage medium for generating a survival probability for an individual diagnosed with metastatic pancreatic cancer, the computer-readable storage medium comprising computer executable instructions which, when executed cause a processing system to execute steps comprising: receiving input data for an individual diagnosed with pancreatic cancer, the input data comprising data for one or more factors of a set of factors; processing the input data to determine one or more numerical measures; and applying a numerical model associated with a predetermined period of time to the one or more numerical measure the numerical model including one or more factors and one or more associated first weighting factor, the one or more factors receiving a value of the one or more numerical value.

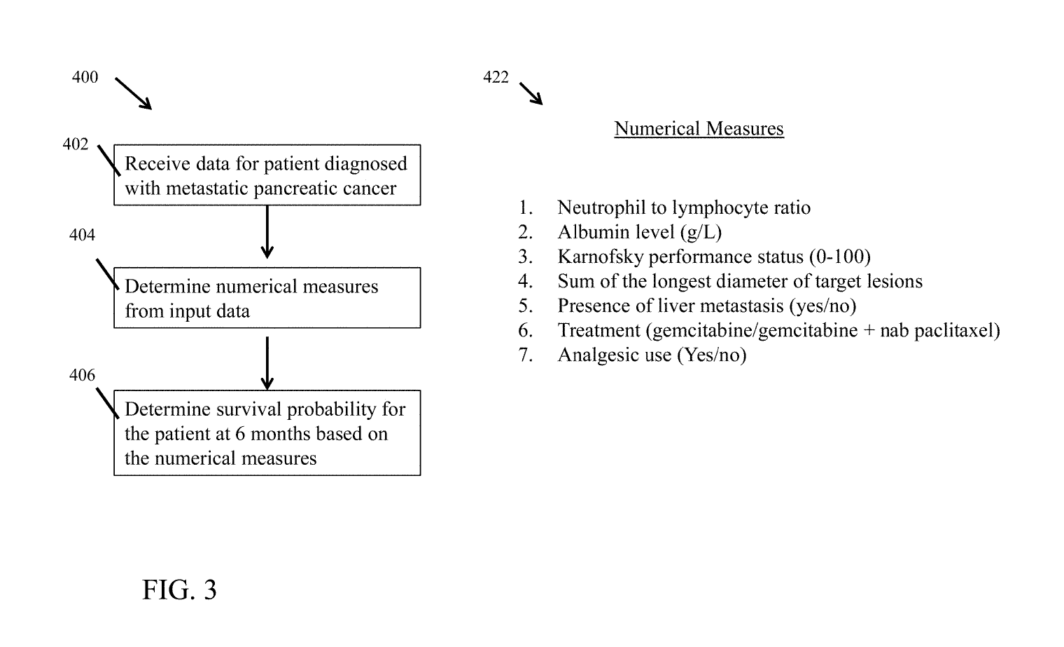

FIG. 3 depicts a flowchart 400 including exemplary steps for generating a 6 month survival probability for an individual diagnosed with metastatic pancreatic cancer. This figure further depicts exemplary numerical measures 422 determined from the patient's input data and used in generating the probability. At 402, input data for a patient diagnosed with metastatic pancreatic cancer is received, where the input data comprises data for multiple factors of a set of patient factors. At 404, one or more numerical measures are determined by processing the input data. The one or more numerical measures may include numerical measures from the exemplary numerical measures 422 of FIG. 3. Additional numerical measures not included in the numerical measures 422 of FIG. 3 may be used in other examples. At 406, a 6 month survival probability is determined by applying the numerical computer model to the determined numerical measures.

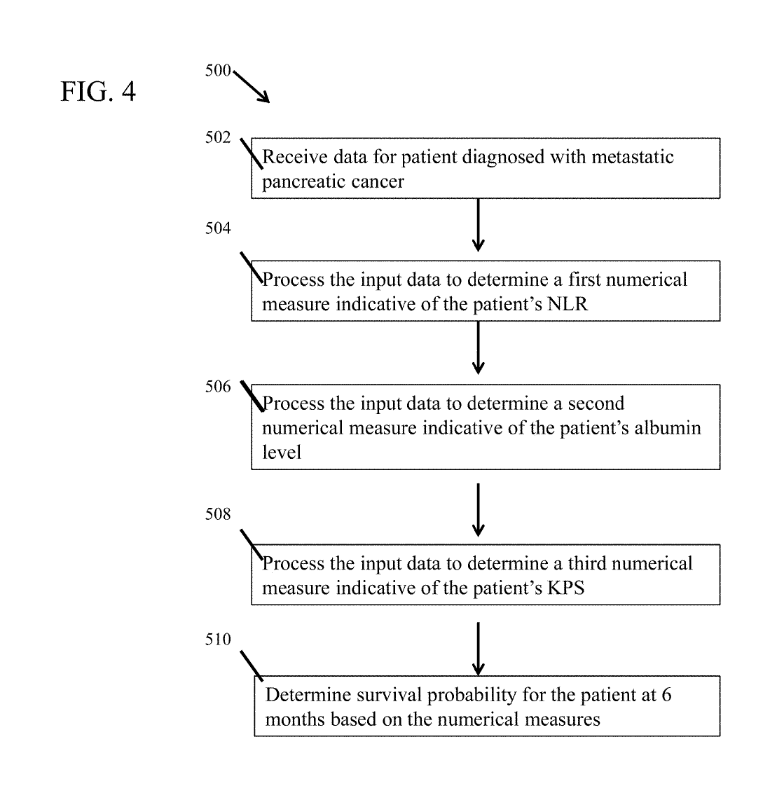

FIG. 4 is a flowchart depicting steps of an exemplary method for generating a survival probability for a patient diagnosed with metastatic pancreatic cancer. At 502, input data for a patient diagnosed with metastatic pancreatic cancer is received. The input data comprises data for multiple factors of a set of patient factors. At 504, the input data is processed to determine a first numerical measure indicative of the patient's NLR. At 506, the input data is processed to determine a second numerical measure indicative of the patient's albumin level. At 508, the input data is processed to determine a third numerical measure indicative of the patient's KPS.

At 510, a numerical computer model associated with a predetermined period time is applied to the first numerical measure, the second numerical measure, and the third numerical measure to determine a probability that the survive within the predetermined period of time. The numerical computer model includes a first factor and an associated first weighting factor, the first factor receiving a value of the first numerical measure. The numerical computer model also includes a second factor and an associated second weighting factor, the first factor receiving a value of the second numerical measure. The numerical computer model further includes a third factor and an associated third weighting factor, the third factor receiving a value of the third numerical measure. The application of the numerical computer model at this stage may involve the actual factor selection, training and configuration of the computer model. Alternatively, the application of the numerical computer model at this stage may involve accessing pre-calculated results the numerical computer model and applying rule-based selection criteria based on the particular numerical measures to select the corresponding mortality value(s) applicable from pre-calculated data from the numerical computer model applicable to the particular numerical measures for the associated factors.

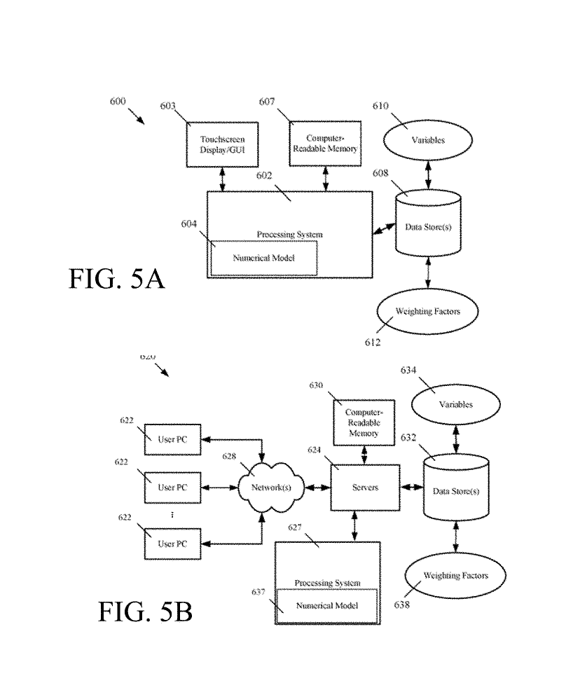

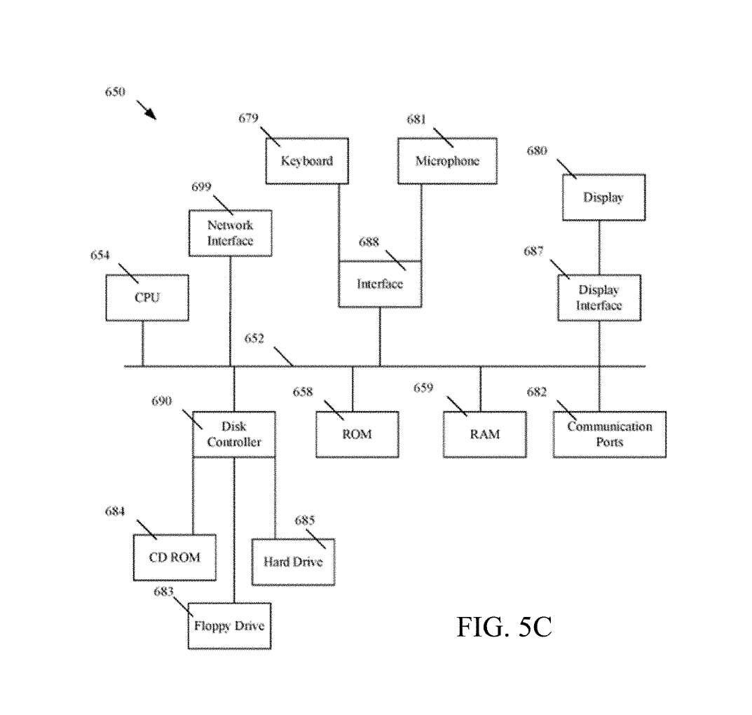

FIGS. 5A-5C depict exemplary systems for implementing the techniques described herein. For example, FIG. 5A depicts an exemplary system 600 that includes a standalone computer architecture where a processing system 602 (e.g., one or more computer processors located in a given computer or in multiple computers that may be separate and distinct from one another) includes a numerical computer model 604 being executed on the processing system 602. For instance, the processing system 602 represented in FIG. 4A may be that of a touchscreen smartphone, a touchscreen tablet, a laptop PC, a desktop PC, etc. Accordingly, the processing system 602 may communicate with a touchscreen display or GUI 603 to display outputs to the user and receive inputs from the user. The processing system 602 has access to a computer-readable memory 607 in addition to one or more data stores 608. The one or more data stores 608 may include factors 610 as well as weighting factors 612. The processing system 602 may be a distributed parallel computing environment, which may be used to handle very large-scale data sets.

FIG. 5B depicts a system 620 that includes a client-server architecture. One or more user PCs 622 access one or more servers 624 running a numerical computer model 604 on a processing system 627 via one or more networks 628. The one or more servers 624 may access a computer-readable memory 630 as well as one or more data stores 632. The one or more data stores 632 may include factors 634 as well as weighting factors 638.

FIG. 5C shows a block diagram of exemplary hardware for a standalone computer architecture 650, such as the architecture depicted in FIG. 5A that may be used to include and/or implement the program instructions of system embodiments of the present disclosure. A bus 652 may serve as the information highway interconnecting the other illustrated components of the hardware. A processing system 654 labeled CPU (central processing unit) (e.g., one or more computer processors at a given computer or at multiple computers), may perform calculations and logic operations required to execute a program. A non-transitory processor-readable storage medium, such as read only memory (ROM) 658 and random access memory (RAM) 659, may be in communication with the processing system 654 and may include one or more programming instructions for performing methods (e.g., algorithms) for constructing a numerical computer model to generate a survival probability for a patient diagnosed with metastatic pancreatic cancer. Optionally, program instructions may be stored on a non-transitory computer-readable storage medium such as a magnetic disk, optical disk, recordable memory device, flash memory, or other physical storage medium.

In FIGS. 5A, 5B, and 5C, computer readable memories 607, 630, 658, 659 or data stores 608, 632, 683, 684 may include one or more data structures for storing and associating various data used in the exemplary systems for constructing a numerical computer model to generate a survival probability for an individual diagnosed with metastatic pancreatic cancer. For example, a data structure stored in any of the aforementioned locations may be used to store data relating to factors and/or weighting factors. A disk controller 690 interfaces one or more optional disk drives to the system bus 652. These disk drives may be external or internal floppy disk drives such as 683, external or internal CD-ROM, CD-R, CD-RW or DVD drives such as 684, or external or internal hard drives 685. As indicated previously, these various disk drives and disk controllers are optional devices.

Each of the element managers, real-time data buffer, conveyors, file input processor, database index shared access memory loader, reference data buffer and data managers may include a software application stored in one or more of the disk drives connected to the disk controller 690, the ROM 658 and/or the RAM 659. The processor 654 may access one or more components as required.

A display interface 687 may permit information from the bus 652 to be displayed on a display 680 in audio, graphic, or alphanumeric format. Communication with external devices may optionally occur using various communication ports 682.

In addition to these computer-type components, the hardware may also include data input devices, such as a keyboard 679, or other input device 681, such as a microphone, remote control, pointer, mouse and/or joystick. Such data input devices communicate with the standalone computer architecture 650 via an interface 688, in some embodiments. The standalone computer architecture 650 further includes a network interface 699 that enables the architecture 650 to connect to a network, such as a network of the one or more networks 628.

Additionally, the methods and systems described herein may be implemented on many different types of processing devices by program code comprising program instructions that are executable by the device processing subsystem. The software program instructions may include source code, object code, machine code, or any other stored data that is operable to cause a processing system to perform the methods and operations described herein and may be provided in any suitable language such as C, C++, JAVA, for example, or any other suitable programming language. Other implementations may also be used, however, such as firmware or even appropriately designed hardware configured to carry out the methods and systems described herein.

The systems' and methods' data (e.g., associations, mappings, data input, data output, intermediate data results, final data results, etc.) may be stored and implemented in one or more different types of computer-implemented data stores, such as different types of storage devices and programming constructs (e.g., RAM, ROM, Flash memory, flat files, databases, programming data structures, programming variables, IF-THEN (or similar type) statement constructs, etc.). It is noted that data structures describe formats for use in organizing and storing data in databases, programs, memory, or other computer-readable media for use by a computer program.

The computer components, software modules, functions, data stores and data structures described herein may be connected directly or indirectly to each other in order to allow the flow of data needed for their operations. It is also noted that a module or processor includes but is not limited to a unit of code that performs a software operation, and can be implemented for example as a subroutine unit of code, or as a software function unit of code, or as an object (as in an object-oriented paradigm), or as an applet, or in a computer script language, or as another type of computer code. The software components and/or functionality may be located on a single computer or distributed across multiple computers depending upon the situation at hand.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language.

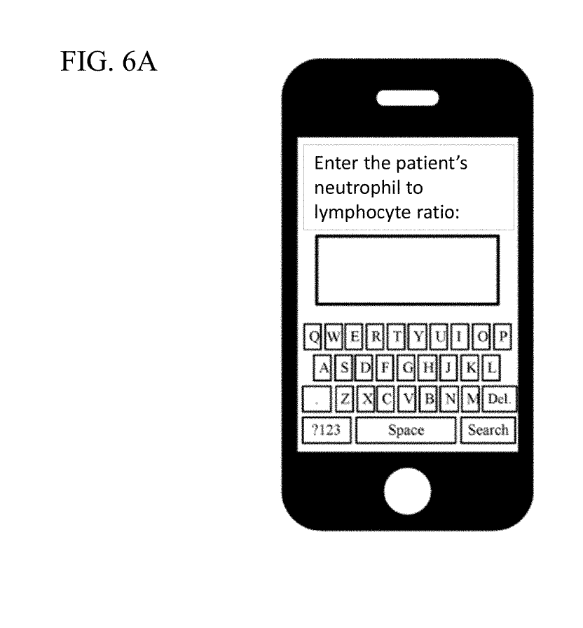

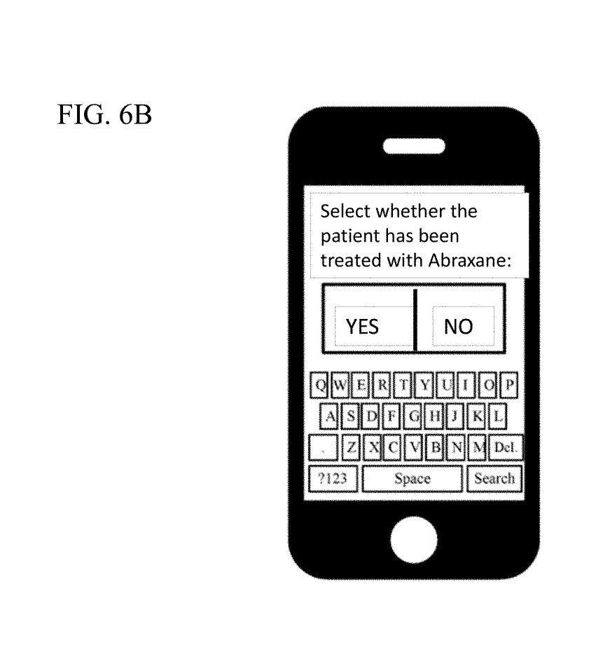

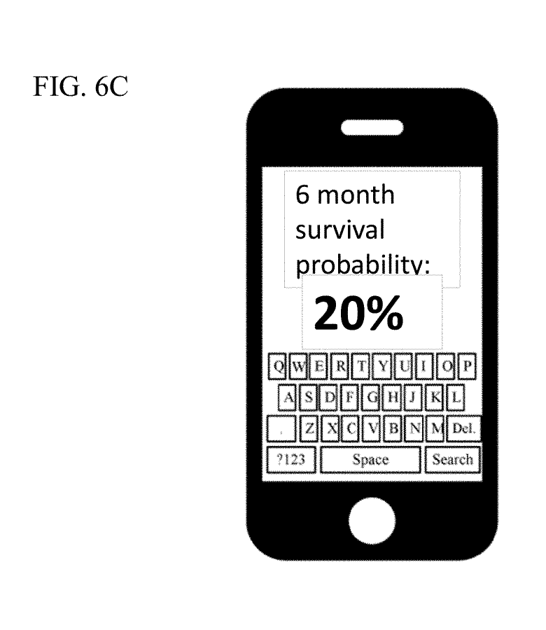

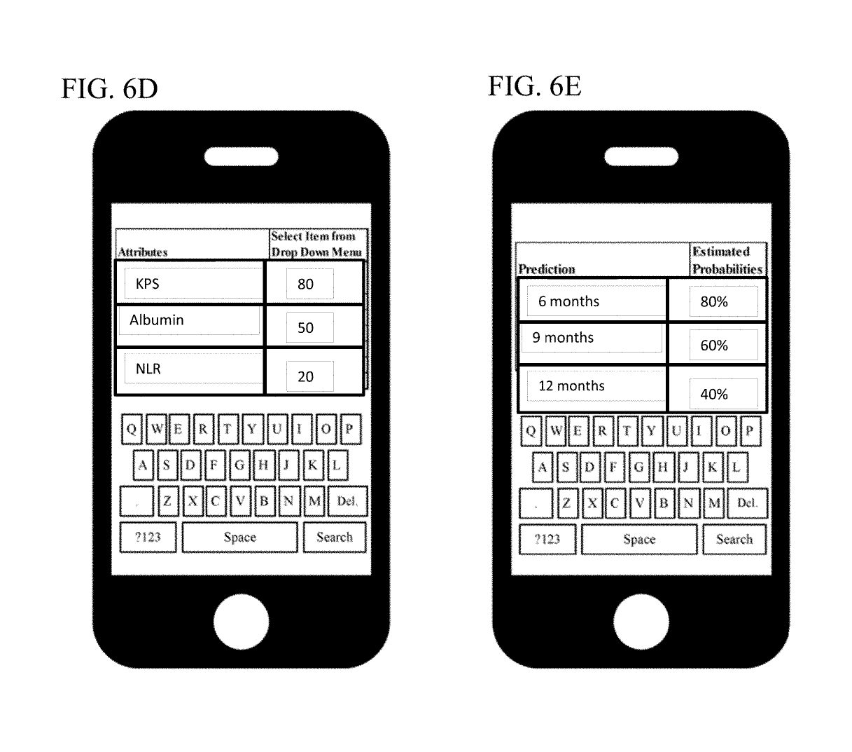

In embodiments of the present disclosure, input data for a patient diagnosed with metastatic pancreatic cancer may be received via a GUI of a software application and based on the computer implemented systems and methods described here, the software application generates a survival probability at a given time period. To illustrate exemplary GUIs for such a software application, reference is made to FIGS. 6A-6C. As illustrated in FIG. 6A, in some embodiments, a GUI prompts a user to provide for various factors. In FIG. 6A, for instance, the GUI prompts the user to “Enter the patient's neutrophil to lymphocyte ratio” and provides a text box for receiving an input from the user. In FIG. 6B, the GUI prompts the user to select whether the patient has been treated with Abraxane (a nab-paclitaxel composition) and provides two buttons for receiving an input from the user. Based on these inputs and inputs for multiple other factors (KPS, analgesic use, albumin level, etc.) received from the user, the software application applies the trained numerical computer model and generates and displays a survival probability. For instance, as show in FIG. 6C, after receiving inputs from the user for multiple factors, the software application generates and displays the survival probability (e.g., “6 month survival probability: 20%, in FIG. 6C).

FIG. 6D illustrates another exemplary GUI for receiving input data representative of factors for a patient diagnosed with metastatic pancreatic cancer. In this example, multiple factors are displayed and for each factor there is a corresponding drop-down menu with multiple selectable options. Although three factors are illustrated in the example of FIG. 6D, it is noted that these factors are examples only, and that in other embodiments a different set of factors may be presented to the user. Based on input data received via that multiple drop-down menus, the software application generates and displays output data on predicted patient mortality. For example as shown in FIG. 6E, after receiving data, the software application generates a table with estimated probabilities for various amounts of time (e.g., 6 months, 9 months, and 12 months).

In some embodiments, the present invention comprises a multivariable COX model comprising multiple factors, wherein the factors are assigned points to the weighted sum of relative significance of each factor.

Methods of Generating a Nomogram

To generate a nomogram used, a model generation module may be used. The model generation module receives the reference data and uses the reference data to determine the weighting factors for the model, e.g. using one or more regression analyses, imputation procedures used to add data that is missing from the reference data, and a model training procedure, all of which are discussed further below. In some embodiments, the reference data is data for a plurality of patients diagnosed with pancreatic cancer. Specifically, in some embodiments, the reference data includes (i) data for multiple variables of a set of patient variables, and (ii) survival data indicative of an amount of time between the patient's pancreatic cancer and the patient's death or between the diagnosis date and the date at which the patient is last known to be alive. The survival data of the reference data spans a range of amounts of time and the reference data is acceptable to train the computer model, or nomogram.

In some embodiments, the weighting factors of the nomogram or numerical computer model are determined via a machine learning application trained based on the reference data. Specifically, the machine learning application may be a logistic regression classifier or a Cox regression classifier. The model generation module performs various procedures (e.g. imputation procedures to add data that is missing from the reference data), in some embodiments, in order to generate the weighting factors of the model. The model generation module provides the model to the probability generating engine, and the probability generating engine uses that model to generate the probability.

With the trained numerical computer model in place, the patient data may be scored by applying the numerical computer model as described above. The probability for the patient data is a probability that the patient will die within a predetermined period of time. In embodiments, the probability generating engine implements multiple models, where each model is associated with a particular period of time. For instance, in an embodiment, the probability generating engine utilizes a first numerical computer model to generate a probability that a patient will die within 6, 9, or 12 months.

Multiple candidate computer models comprising different combinations of the variables of the set of patient variables are generated. Each of the candidate computer models includes multiple weighting factors associated with the variables, and each variable of each candidate computer model has an associated weighting factor. Multiple computerized numerical regression analyses for the multiple candidate computer models are conducted based on the data for the variables and the survival data to determine first selected variables and second selected variables from the set of patient variables. The first selected variables satisfy one or more selection criteria to be deemed predictive of mortality for a first predetermined period of time (e.g., mortality within 6, 9, 12 months from diagnosis) for patients diagnosed with pancreatic cancer.

In embodiments, performing begins with univariate screening to reduce the number of variables and then proceeds to a variable selection procedure. Specifically, in embodiments, univariate analyses are conducted with the intent of determining the degree of missingness on each variable and the statistical significance of the variable in predicting the dependent measure (e.g., death within a predetermined period of time). In some embodiments, variables significant at the p>0.15 level and with less than 60% missing data are screened in.

In embodiments, in building the first computer model used to generate a probability that a patient diagnosed with pancreatic cancer will die within 180 days, the univariate analyses are logistic regression analyses conducted for the discrete variable of mortality within 180 days. Exemplary SAS code for the logistic regression analyses follows, where d 180 is the discrete dependent variable:

proc logistic data=Edeath descending;

model d180=&var/risklimits;

ods output ParameterEstimates=&univ est NObs &univ miss;

run;

By contrast, in building the second computer model used to generate a probability that a patient diagnosed with pancreatic cancer will die within 1 year, 2 years, 3 years, or 4 years, the univariate analyses are Cox regression analyses, in embodiments. In embodiments, the Cox regression analyses are used to handle censored data. Data is censored when patients discontinue or are otherwise lost to follow-up. From such data, it cannot be determined if the patients are currently dead or alive, and the data merely indicates that after a certain duration of follow-up, the patient discontinued follow-up or was otherwise lost to follow-up.

To address the issue of missing data in the reference data, a number of imputed datasets are created, in embodiments. The relative efficiency (RE) of multiple imputation is given by the following:

RE=(1+λ/m)⁻¹,

where .A is the fraction of missing information about the parameter being estimated, and m is the number of imputed datasets. The fraction of missing data is roughly proportional to the average amount of missing data.

In embodiments, Rubin's imputation framework may be used for the imputation analysis. This analysis involves (i) assuming an imputation model, (ii) obtaining the predictive distribution of the missing data conditional on observed data and distribution parameters, and (iii) producing multiple imputed datasets using the predictive distribution. Analysis under multiple imputation is robust under less restrictive assumptions of Missing at Random (MAR) compared to the case-wise deletion of data records with any data missing on any variable. Further, case-wise deletion of data missing on any variable leads to considerable loss of information on other collected variables. In embodiments, the imputation model utilized is the Markov Chain Monte Carlo (MCMC) method under the multivariate normal model. All variables (including those screened out) are used in the imputation model to extract all information on the missingness of the predictors contained in the dataset, and ten imputations are generated, in embodiments. Exemplary SAS code for performing this analysis is as follows:

proc mi data=Edeath nimpute=10 seed=651467 out=Edeathm var agen hispan bmi issstagen mhecogynn . . . partial list of variables

run;

In embodiments, following the univariate screening and imputation procedures described above, a computer-implemented variable selection procedure is performed. In the variable selection procedure, the imputed datasets are stacked on top of each other, and the multivariate logistic and Cox regressions are run using underweighted observations with the underweighting being proportional to the number of imputed datasets and to the degree of missingness. The variables used are those screened in under the univariate regression analyses described above. The SAS code for the first computer model (e.g., the logistic model, as described herein) requesting all possible models follows. The weight is equal to (1−f)/(#of imputations), where f is the average fraction of missing data.

proc logistic data=Edeathm2;

model d180 (event=‘yes’)=agen issstagen mhecogynn imwg_risk mhdiabn mhhyn calcium creat plat_ct caref mobf gp_17p_ad novelf/

selection=score details lackfit; weight wt;

run;

The code “selection=score” provides the score statistic for all possible models. In embodiments, the difference in score statistics between models is a chi-squared distribution with degrees of freedom given by the difference in the number of variables in the models. In embodiments, starting with the best I-variable model, movement in one variable increments to the best k-variable model is performed until the incremental score statistic is less than the critical value obtained as the 0.1-level Wald X2 chi-square value for one degree of freedom. In embodiments, a number of models with score statistics in the neighborhood of that for the best k-variable model are considered, and the most clinically appropriate model is selected.

In embodiments, in building the first computer model for generating a probability that a patient diagnosed with pancreatic cancer, the variable selection procedure described above may result in the selection of six, sever, or eight variables (or factors). As described herein, these variables are selected using a stacked, weighted logistic regression analyses.

The training of the computer model may include (i) processing the reference data to determine, for patients represented in the reference data, numerical measures for respective variables of the first selected variables, and (ii) conducting a first computerized numerical regression analysis based on the determined numerical measures to determine the first weighting factors. Likewise, the training of the second computer model may include (i) processing the reference data to determine, for patients represented in the reference data, numerical measures for respective variables of the second selected variables, and (ii) conducting a second computerized numerical regression analysis based on the determined numerical measures to determine the second weighting factors. For example, in an embodiment in which the first or second selected variables include a variable indicative of an age of the patient, the reference data is processed to determine, for respective patients represented in the reference data, numerical values corresponding to the patients' ages. Likewise, in an embodiment in which the first or second selected variables include a variable indicative of a stage of the patient's pancreatic cancer, the reference data is processed to determine, for respective patients represented in the reference data, numerical values corresponding to disease stages. After determining the numerical measures, the aforementioned numerical regression analyses are conducted based on the numerical measures and survival data for the respective patients represented in the reference data to determine the weighting factors of the respective first and second computer models.

In embodiments, a machine learning approach is used to build and train the computer models. In constructing the computer model, the determined numerical measures may be combined in a logistic regression classifier, which uses the determined numerical measures and the survival data for the patients represented in the reference data to generate weighting factors for the numerical measures. In constructing the computer model, the determined numerical measures may be combined in a Cox regression classifier, which uses the determined numerical measures and the survival data for the patients represented in the reference data to generate weighting factors for the numerical measures.

The computer model is updated to include the determined numerical values for the first weighting factors and the second weighting factors for each selected variable of the first and second selected variables. Accordingly, the computer model is configured to generate probability data that a patient satisfying certain first selectable criteria will die within the first predetermined period of time (e.g., 3, 6, or 9 months). The computer model is then ready to be used for generating probabilities, i.e., to receive numerical measures corresponding to variables of the respective computer models, where the numerical measures are new data for a patient, so as to generate a probability that the patient will die within the a predetermined periods of time. In this manner, the numerical computer models are thereafter configured to perform automated determination of probabilities for new patient data.

As described above, in some embodiments, a prediction matrix is generated, and the prediction matrix includes probability values for all possible combinations of patient input data. The above steps are used to generate a blank matrix with column and row headers, in embodiments. To populate these blank cells with the appropriate probability values, the numerical computer model is used to compute the probabilities for every possible combination of patient input values. The probabilities are then inserted into the prediction matrix.

The generation of an exemplary prediction matrix will now be described. Steps similar to those described above for generating a blank matrix are used. To populate these blank cells with appropriate probability values, the numerical computer model is used to compute the probabilities for every possible combination of patient input values.

Exemplary SAS code to implement this starts with SAS PROC PLAN code, and a dataset “covals” is generated. This dataset contains the combinations of the levels of the predictors along with the mapping to cells in the matrix. To generate the probabilities for filling the matrix, the exemplary code below uses the covals dataset in the baseline statement of the SAS PHREG procedure to generate survival probabilities at every event time in the registry along with confidence intervals. To obtain the survival probability beyond three years, the data records corresponding to event time closest to and less than the three-year time-point (1095 days) are retained. The prediction of survival beyond three years for each predictor combination is estimated as the average of the corresponding 3 year survivals from each of the imputations.

Computer models are validated. Each of the computer models may be validated with both an “internal” validation procedure and an “external” validation procedure. The validation of the first computer model used in generating a probability that a patient diagnosed with pancreatic cancer will die within 3, 6, 9, or 23 months will now be described. In some embodiments, internal validation involves the splitting of the dataset into test and training samples, and the model obtained in the training sample is evaluated in the test sample. Better estimates of validation indices may be obtained when they are obtained through analysis of repeated random splits into test and training samples, a process referred to as bootstrap re-sampling. The validation index used in embodiments to measure the predictive ability of the computer model is Harrell's C-Index. This index is interpretable as a concordance probability, i.e., the probability that a randomly selected pair of patients, one with a poorer survival outcome than the other, will be correctly differentially identified based on inputting the two patients' baseline prognostic characteristics in the fitted model.

### EXAMPLE 1

The large phase 3 MPACT trial (N=861) provided a robust dataset for the development of a nomogram to predict overall survival in patients with metastatic pancreatic cancer treated with chemotherapy(Von Hoff D D, Ervin T, Arena F P et al. Increased survival in pancreatic cancer with nab-paclitaxel plus gemcitabine. N Engl J Med 2013; 369: 1691-1703). In MPACT, patients were randomized to receive either nab-paclitaxel plus gemcitabine or gemcitabine alone as first-line treatment. The median follow-up for overall survival (OS) across both treatment arms was 13.9 months, and the combination of nab-paclitaxel plus gemcitabine demonstrated a significantly longer OS vs gemcitabine alone (median, 8.7 vs 6.6 months; HR 0.72; 95% confidence interval [CI], 0.62 to 0.83, P<0.001) (Goldstein D, El-Maraghi R H, Hammel P et al. nab-Paclitaxel plus gemcitabine for metastatic pancreatic cancer: long-term survival from a phase III trial. J Natl Cancer Inst 2015; 107: 10.1093/jnci/dju413. Print 2015 Feb). Multivariable analyses have been conducted to determine which factors were independently predictive of survival in the MPACT study; however, these analyses did not allow for individualized patient prediction. (Von Hoff D D, Ervin T, Arena F P et al. Increased survival in pancreatic cancer with nab-paclitaxel plus gemcitabine. N Engl J Med 2013; 369: 1691-1703; Goldstein D, El-Maraghi R H, Hammel P et al. nab-Paclitaxel plus gemcitabine for metastatic pancreatic cancer: long-term survival from a phase III trial. J Natl Cancer Inst 2015; 107: 10.1093/jnci/dju413. Print 2015 February Ballehaninna U K, Chamberlain R S. Serum C A 19-9 as a biomarker for pancreatic cancer—a comprehensive review. Indian journal of surgical oncology 2011; 2: 88-100.)

### Methods

MPACT Study design

The design and patient characteristics of the phase 3, open-label, randomized MPACT study have been described previously (Von Hoff D D, Ervin T, Arena F P et al. Increased survival in pancreatic cancer with nab-paclitaxel plus gemcitabine. N Engl J Med 2013; 369: 1691-1703). Eligible patients were randomized (1:1 ratio; stratified by KPS, presence of liver metastases, and geographic region) to receive either nab-paclitaxel plus gemcitabine or gemcitabine alone until disease progression by RECIST or unacceptable toxicity. All independent ethics committees at each participating institution approved the trial, which was conducted in accordance with the International Conference on Harmonisation E6 requirements for Good Clinical Practice.

### Patient Population

Patients with metastatic adenocarcinoma of the pancreas, Karnofsky performance status ≥70 and bilirubin level ≤upper limit of normal were included in the study. Patients were excluded if they had received prior chemotherapy in the adjuvant or metastatic setting (5-fluorouracil or gemcitabine was allowed as sensitizers for radiation therapy).

### Statistical Analyses

A total of 34 factors were chosen to be included in the univariable analyses of overall survival. Two of the factors (metastases of the brain and the extremities) were excluded because the values were constant (i.e., 0 for all patients), which resulted in 32 factors tested in the univariable analysis. The following 7 baseline demographic factors were included: age, gender, race/ethnicity, height, weight, body mass index (BMI), and body surface area (Table 3). In addition, 25 clinical factors were analyzed (Table 3).

TABLE 3
Univariable candidate predictor factors and multivariable Cox proportional hazard model to
predict survival.

| | Univariable analysis | | | Multivariable analysis | | |
|---|---|---|---|---|---|---|
| Baseline Factors^{a} | HR | 95% CI | P value^{a} | HR | 95% CI | P value |

Factor

| | | | | | | |
|---|---|---|---|---|---|---|
| Neutrophil to | 1.07 | 1.06-1.09 | <0.001 | 1.05 | 1.04-1.07 | <.001 |
| lymphocyte ratio | | | | | | |
| Albumin level (g/L) | 0.93 | 0.92-0.94 | <0.001 | 0.94 | 0.92-0.95 | <.001 |
| Karnofsky performance | 0.96 | 0.95-0.97 | <0.001 | 0.97 | 0.96-0.98 | <.001 |
| status | | | | | | |
| Sum of the longest | 1.03 | 1.02-1.04 | <0.001 | 1.02 | 1.01-1.03 | <.001 |
| diameter of target | | | | | | |
| lesions (cm) | | | | | | |
| Presence of liver | 1.79 | 1.44-2.22 | <0.001 | 1.62 | 1.29-2.04 | <.001 |
| metastases | | | | | | |
| Treatment arm | | | | | | |
| nab-paclitaxel plus | Reference | — | <0.001 | Reference | — | <.001 |
| gemcitabine | | | | | | |
| Gemcitabine alone | 1.36 | 1.17-1.58 | | 1.56 | 1.34-1.82 | |
| Analgesic use | 1.16 | 1.00-1.35 | 0.048 | 1.16 | 0.99-1.36 | .07 |
| CA19-9 level^{b} | 1.00 | 1.00-1.00 | 0.004 | — | — | — |
| Number of metastatic | 1.14 | 1.05-1.23 | 0.002 | — | — | — |
| sites | | | | | | |
| Localization of | | | | | | |
| pancreatic tumor | | | | | | |
| Body | Reference | — | 0.041 | — | — | — |
| Head | 1.05 | 0.88-1.26 | | | | |
| Tail | 1.35 | 1.11-1.64 | | | | |
| Presence of biliary stent | 0.96 | 0.79-1.16 | 0.66 | — | — | — |
| Presence of peritoneum | 1.35 | 1.03-1.78 | 0.026 | — | — | — |
| metastases | | | | | | |
| Prior chemotherapy | 0.56 | 0.38-0.83 | 0.002 | — | — | — |
| Prior radiation therapy | 0.63 | 0.41-0.94 | 0.013 | — | — | — |
| Prior Whipple | 0.64 | 0.48-0.86 | 0.001 | — | — | — |
| procedure | | | | | | |

Demographic Factors

| | | | | | | |
|---|---|---|---|---|---|---|
| Age | 1.01 | 1.00-1.01 | 0.052 | — | — | — |
| BMI | 1.00 | 0.98-1.01 | 0.64 | — | — | — |
| Race/ethnicity | | | | | | |
| Asian | Reference | — | 0.17 | — | — | — |
| Black | 1.57 | 0.77-3.18 | | | | |
| Hispanic | 1.93 | 1.01-3.69 | | | | |
| White | 1.80 | 1.00-3.25 | | | | |
| Other | 2.69 | 1.07-6.76 | | | | |
| Sex | | | | | | |
| Female | Reference | — | 0.11 | — | — | — |
| Male | 1.13 | 0.97-1.31 | | | | |
| Weight | 1.00 | 1.00-1.01 | 0.71 | — | — | — |

For the sum of longest tumor diameters, ≤10 target lesions (maximum of 5 per organ) were selected; generally the largest, most reliably measured, and most representative of the patient's sites of disease were chosen. For continuous variables, missing data were replaced with the mean from the non-missing data. For the continuous variable CA19-9, the upper outliers (>75^{th} percentile+1.5×interquartile range) were assigned the 95^{th} percentile value. For discrete variables, missing data were assigned the new category level of missing. For CA19-9, separate analyses were carried out for patients that did or did not have baseline CA19-9 values; patients without CA19-9 values were either CA19-9 non-secretors (non-expressers) or were missing baseline values. CA19-9 was not retained in the multivariate analysis (see below) after backward selection; therefore, the final Cox model included all patients, regardless of whether or not they expressed CA19-9.

Univariable Cox analyses were used to assess each of the 32 factors' association with overall survival. Factors that were associated with overall survival at P<0.1 or that were of known clinical importance were carried forward to a Cox multivariate model. To remain in the multivariate model, factors had to remain significantly associated with overall survival at the P<0.1 level after backward selection. Factors identified in the multivariate model were used to develop a nomogram which assigned points equal to the weighted sum of the relative significance of each factor. The factor that was the most predictive was assigned a maximum point value of 100, and other factors' points were determined based on comparison with this most influential factor.

After creating the primary nomogram, the effect of individually adding 5 factors that were not statistically predictive, but were believed to be clinically important (CA19-9, age, number of metastatic sites, number of lesions, and lung metastasis), was examined to determine how much these factors would contribute to the predictive ability of the nomogram if forced into the model. For the analysis of CA19-9, patients with missing values and non-secretors were excluded.

All nomograms were internally validated using bootstrapping (with 1000 iterations), a concordance index (c-index), and calibration plots used to discriminate low-, intermediate-, and high-risk groups. The three risk groups were created using a risk stratification method in which the nomogram scores from all patients were split into 4 quartiles; the first quartile constituted the low-risk group, the middle 2 quartiles the intermediate-risk, and the fourth quartile the high-risk group. The resampling model calibration used bootstrapping to obtain bias-corrected estimates of predicted vs observed values based on categorizing predictions into 5 intervals. A single summary value was reported by taking the mean of the 5 interval values.

### Results

### Patients

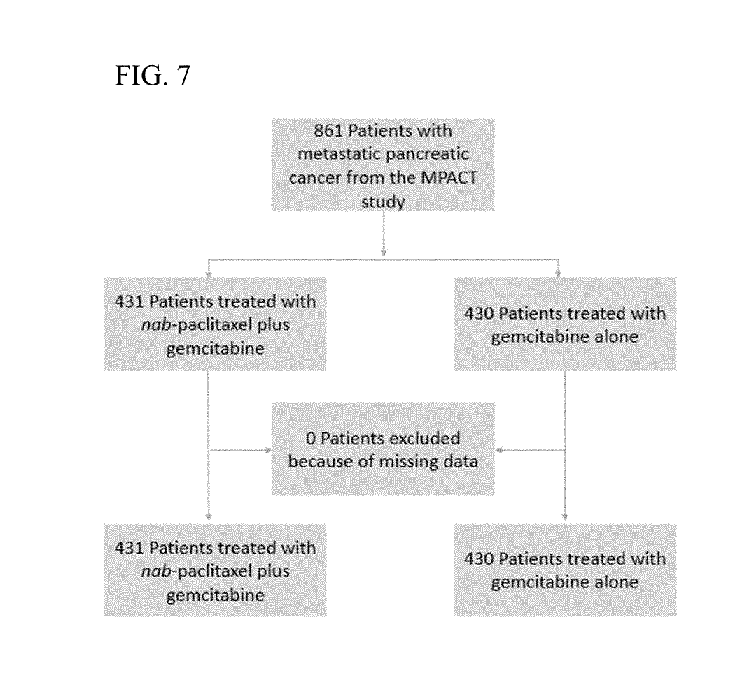

Data from 861 patients (nab-paclitaxel plus gemcitabine, n=431; gemcitabine alone, n=430) enrolled in the MPACT study were included in this analysis (FIG. 7).

### Univariable and Multivariable Models

Fourteen out of a total of 32 factors examined in univariable analyses of overall survival were determined to be statistically significantly associated with survival (Table 3). In addition, 6 factors were chosen to proceed to a multivariate analysis because of clinical relevance and/or their close proximity to the prespecified alpha-level (P<0.1): age, BMI, presence of biliary stent, race/ethnicity, sex, and weight. Out of the 20 factors entered into the multivariate model, 7 factors remained after backward selection and were identified as being significantly associated with overall survival (Table 3).

Primary Nomogram with Internal Validation

A nomogram was generated using the 7 factors identified by multivariate analysis (FIG. 1) and was shown to predict the survival probabilities at 6, 9, and 12 months. For example, a patient receiving nab-paclitaxel plus gemcitabine (0 points) with a baseline albumin level of 50 (16 points), who is using analgesics (4 points), has a Karnofsky performance status score of 80 (14 points), a neutrophil-to-lymphocyte ratio of 20 (25 points), with no liver metastases (0 points), and a sum of longest diameter of tumors of 10 cm (4 points) has a total score of 63, which corresponds to 6-, 9-, and 12-month predicted survival probabilities of 66%, 46%, and 32%, respectively (Table 1).

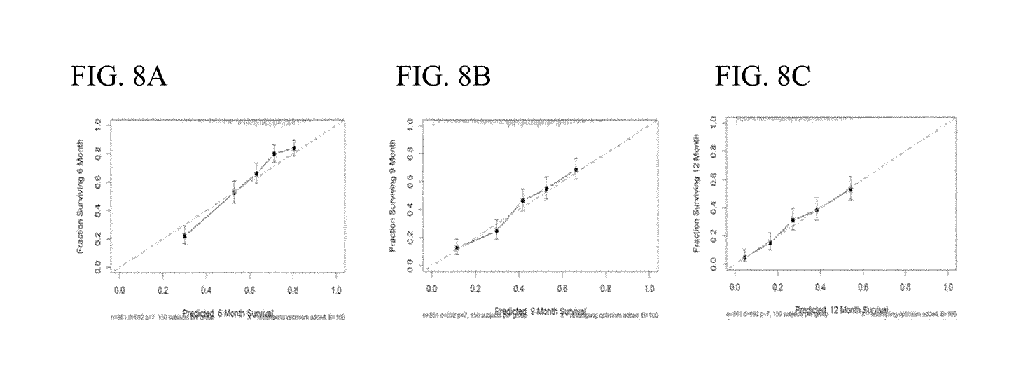

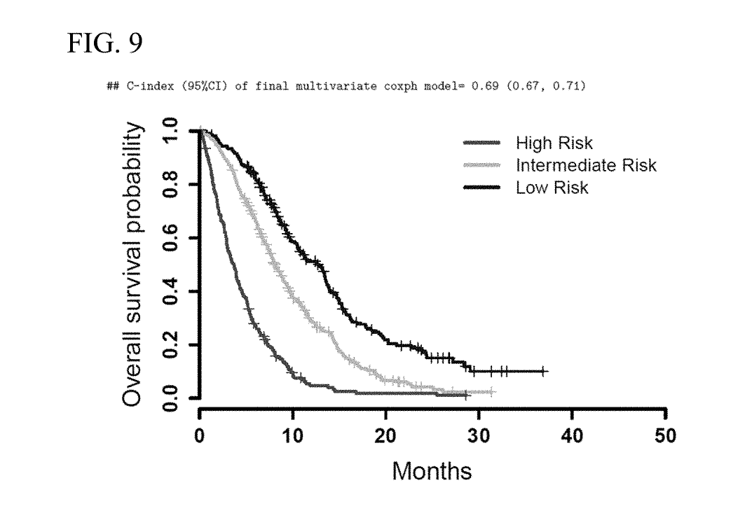

In calibration plots, the mean absolute errors between the observed and predicted probabilities for 6-, 9-, and 12-month survival were 0.04, 0.03, and 0.01, respectively (FIGS. 7A-7C). The nomogram was able to distinguish low—(n=216), intermediate—(n=430), and high—(n=215) risk groups (c-index 0.69; 95% CI, 0.67-0.71) which had median overall survival values of 12.9, 8.2, and 3.7 months, respectively (FIG. 8).

### Relative Contribution of Clinically Important Factors Added Individually to Primary Nomogram

In analyses that forced each of the 5 clinically important factors individually to the primary nomogram, it was demonstrated that CA19-9, number of metastatic sites, and lung metastasis individually only contributed up to 1 point; number of lesions contributed up to 10 points, and age contributed up to 7 points (Table 4). The Akaike information criterion (AIC) of the final nomogram model was 7918, which was lower and thus reflective of greater predictive power than models in which the following factors were added: age (AIC=7919), number of baseline lesions (AIC=7919), metastases to the lung (AIC=7920), or number of metastatic sites (AIC=7920). The AIC for CA19-9 should not be compared with the other models because the CA19-9 analysis was conducted on a smaller set of patients (n=634).

TABLE 4
Relative contribution of factors in a nomogram for prediction of overall survival in patients
with metastatic pancreatic cancer

| | | | Nomograms, Points Contributed per Factor^{a} | | | | | |
|---|---|---|---|---|---|---|---|---|
| | Range per Factor | | | Primary Plus Each of the Below Factors Individually | | | | |
| | Value Worth | Value Worth | | | | Number | | |
| | Most Points | Least Points | | | | of | Number | |
| | (Worse | (Better | | | | Metastatic | of | Lung |
| Factor | Prognosis) | Prognosis) | Primary | CA19-9^{b} | Age | Sites | Lesions | Metastasis |
| NLR | 80 | 0 | 100 | 64 | 100 | 100 | 100 | 100 |
| Albumin, g/L | 10 | 60 | 80 | 100 | 80 | 80 | 80 | 80 |
| KPS | 60 | 100 | 28 | 35 | 28 | 28 | 27 | 28 |
| SLD, cm | 50 | 0 | 19 | 27 | 20 | 19 | 14 | 19 |
| Presence of liver | Yes | No | 12 | 19 | 12 | 12 | 12 | 12 |
| metastasis | | | | | | | | |
| Treatment arm | Gem | nab-P plus | 11 | 19 | 11 | 11 | 11 | 11 |
| | | Gem | | | | | | |
| Analgesic use at | Yes | No | 4 | 7 | 4 | 4 | 4 | 4 |
| baseline | | | | | | | | |
| CA19-9 level, | ≥400,000 | ≤100,000 | — | 1 | — | — | — | — |
| U/mL | | | | | | | | |
| Age, years | 90 | 20 | — | — | 7 | — | — | — |
| Number of | ≥5 | <5 | — | — | — | 1 | — | — |
| metastatic sites | | | | | | | | |
| Number of lesions | 30 | <5 | — | — | — | — | 10 | — |
| Lung metastasis | Yes | No | — | — | — | — | — | 1 |

CA19-9, carbohydrate antigen 19-9;
Gem, gemcitabine;
KPS, Karnofsky performance status;
nab-P, nab-paclitaxel;
NLR, neutrophil-to-lymphocyte ratio;
OS, overall survival;
SLD, sum of longest tumor diameters.
^{a}Points contributed to the nomogram as a measure of the relative importance of each factor; the greater the number, the greater the factor's contribution to the model.
^{b}The CA19-9 nomogram was created using data from a smaller subset of patients (n = 634) because non-secretors (non-expressors) were excluded.

### Discussion

This prognostic nomogram demonstrated that survival at 6, 9, and 12 months could be estimated using baseline factors, including albumin level, neutrophil-to-lymphocyte ratio, Karnofsky performance status, treatment arm, presence of liver metastases, sum of the longest diameter of target lesions, and analgesic use. This nomogram may allow physicians and patients to make more informed and individualized decisions about treatment and management of metastatic pancreatic cancer.

Several multivariate analyses of survival have been conducted on data from the MPACT study, and results are generally consistent despite some variation due to differences in methodology and lists of factors evaluated. (Von Hoff D D, Ervin T, Arena F P et al. Increased survival in pancreatic cancer with nab-paclitaxel plus gemcitabine. N Engl J Med 2013; 369: 1691-1703; Goldstein D, El-Maraghi R H, Hammel P et al. nab-Paclitaxel plus gemcitabine for metastatic pancreatic cancer: long-term survival from a phase III trial. J Natl Cancer Inst 2015; 107: 10.1093/jnci/dju413. Print 2015 February Ballehaninna U K, Chamberlain R S. Serum C A 19-9 as a biomarker for pancreatic cancer—a comprehensive review. Indian journal of surgical oncology 2011; 2: 88-100.)

One such analysis examined a set of factors largely prespecified by the study protocol and found the following to be significantly associated with increased survival: treatment arm (nab-paclitaxel plus gemcitabine vs gemcitabine alone; HR 0.68; 95% CI, 0.57-0.80; P<0.001), presence of liver metastases (HR 1.65; 95% CI, 1.28-2.12; P<0.001), baseline KPS (70-80 vs 90-100; HR 1.47; 95% CI, 1.24-1.74; P<0.001), and neutrophil-to-lymphocyte ratio (not prespecified in the study protocol; HR 0.57; 95% CI, 0.48-0.68; P<0.001). In addition to these factors, the final multivariate analysis in the current study also included the following factors not prespecified by the study protocol: albumin level, the sum of the longest diameter of target lesions, and analgesic use.

The current analysis also identified CA19-9 level as a potential predictive factor at the univariable level; however, the factor did not ultimately remain significant in the final multivariate model. This finding agrees with a previous study by Tabernero and colleagues, which also did not retain CA19-9 as a predictive factor in a multivariate model of survival [9]. When forced into the primary nomogram CA19-9 only contributed up to 1 point. Perhaps, CA19-9 may somehow co-segregate with other factors, which would explain the lack of additional information allowed by forcing it into the primary nomogram. Although CA19-9 is often considered in patient prognosis, its value as a predictive marker is further called into question by the proportion of patients who don't secrete it.

In addition to exploring the potential of adding CA19-9 into the primary nomogram, the present analysis also investigated the inclusion of other clinically relevant factors such as age, number of metastatic sites, number of lesions, and lung metastasis. However, none of these factors contributed substantially to the prognostic information to warrant inclusion in the nomogram.

Currently, physicians who wish to estimate their patients' probability of survival at different time points must rely on averaged statistical data available from large databases, published risk group data, or staging systems that do not allow for individually tailored predictions. The predictive nomogram is a beneficial tool because it allows for a risk prediction specific to each patient. Internal validation of the nomogram demonstrated that it was reliable for the prediction of survival in the low-, intermediate-, and high-risk groups; the estimated survival times were closely aligned with the actual values and the c-index score was 0.69.

A limitation of the present study was that the internal validation method utilized bootstrapping, which is a useful resampling method for reducing the propensity of a model to be overfit to a specific dataset, but cannot ensure that the model will be applicable to an external cohort. The size and breadth of the MPACT dataset, which involved patients from a variety of settings and with a range of performance statuses, may address this lack of an external validation cohort. In addition, the present nomogram includes sum of longest diameter of target lesions and neutrophil-to-lymphocyte ratio, which may be less familiar to some physicians. However, both should be obtainable from existing patient measurements with the potential extra step of calculation. Neutrophil and lymphocyte counts are routinely measured before treatment, and physicians can use a simple algorithm to calculate neutrophil-to-lymphocyte ratio. The sum of longest diameters of target lesions could also be obtained from radiographic scans.

### Conclusions

The present nomogram can be used to predict the survival of patients with metastatic pancreatic cancer treated with nab-paclitaxel plus gemcitabine or gemcitabine alone. A more accurate estimation of survival may guide physicians and patients in their decisions regarding metastatic pancreatic cancer treatment.

### EXAMPLE 2

The objectives of this study analysis were to develop a nomogram to predict overall survival for patients with metastatic pancreatic cancer excluding treatment (i.e. treatment with nab-paclitaxel plus gemcitabine or gemcitabine alone) as a factor, to allow the nomogram to be more generalizable.

### Methods

MPACT Study Design

The design and patient characteristics of the phase 3, open-label, randomized MPACT study have been described previously. In brief, patients with metastatic pancreatic cancer undergoing first-line therapy for their disease were randomly assigned to receive either nab-paclitaxel plus gemcitabine or gemcitabine alone until disease progression by RECIST or unacceptable toxicity. All independent ethics committees at each participating institution approved the trial, which was conducted in accordance with the International Conference on Harmonisation E6 requirements for Good Clinical Practice.

### Patient Population

Patients with metastatic adenocarcinoma of the pancreas, Karnofsky performance status ≥70 and bilirubin level ≤upper limit of normal enrolled in the MPACT study were included in the analyses. In MPACT study patients were excluded if they had received prior chemotherapy in the adjuvant or metastatic setting (5-fluorouracil or gemcitabine was allowed as sensitizers for radiation therapy).

### Nomogram Development and Validation

Univariable Cox proportional hazard model analyses were used to assess each of the 32 factors' association with overall survival. Factors that were associated with overall survival at P<0.1 or that were of known clinical importance were carried forward to a Cox multivariable proportional hazard model. To remain in the multivariable model, factors had to remain significantly associated with overall survival at the P<0.1 level after backward selection. Factors identified in the multivariable model were used to develop a nomogram which assigned points equal to the weighted sum of the relative significance of each factor. The factor that was the most predictive was assigned a maximum point value of 100, and other factors' points were determined based on comparison with this most influential factor.

After creating the primary nomogram, the effect of individually adding 5 factors that were not statistically predictive, but were believed to be clinically important (CA19-9, age, number of metastatic sites, number of lesions, and lung metastasis), was examined to determine how much these factors would contribute to the predictive ability of the nomogram if forced into the model. For the analysis of CA19-9, patients with missing values and non-secretors were excluded.

All nomograms were internally validated using bootstrapping (with 1000 iterations), a concordance index (c-index) to test the ability of the nomogram to distinguish between high versus low risk patients, and calibration plots to determine how accurately the nomogram-estimated risk corresponded to the actual observed risk. Additional statistical methods are provided in supplemental materials.

A total of 34 factors were chosen to be included in the univariable analyses of overall survival. These factors were considered because prior prognostic studies have identified them to be significant. Other factors were considered with no prior studies because they were considered to be of clinical interest amongst the study investigators. Treatment was excluded as a factor of interest to allow the nomogram to be more generalizable. Two factors (metastases of the brain and the extremities) were excluded because the values were constant (ie, 0 for all patients), which resulted in 32 patient and clinical factors tested in the univariable analysis (Table 5).

TABLE 5
Univariable candidate predictor factors and multivariable Cox proportional hazard model
to predict survival.

| | Univariable analysis | | | Multivariable analysis | | |
|---|---|---|---|---|---|---|
| Baseline Factors^{a} | HR | 95% CI | P value^{a} | HR | 95% CI | P value |

Clinical Factors

| | | | | | | |
|---|---|---|---|---|---|---|
| Neutrophil to lymphocyte | 1.07 | 1.09-1.09 | <0.001 | 1.05 | 1.04-1.07 | <.001 |
| ratio | | | | | | |
| Albumin level (g/L) | 0.93 | 0.92-0.94 | <0.001 | 0.94 | 0.93-0.96 | <.001 |
| Karnofsky performance | 0.97 | 0.96-0.97 | <0.001 | 0.98 | 0.97-0.99 | <.001 |
| status | | | | | | |
| Presence of liver metastasis | 1.67 | 1.37-2.05 | <0.001 | 1.44 | 1.17-1.77 | <.001 |
| Sum of the longest diameter | 1.03 | 1.02-1.04 | <0.001 | 1.02 | 1.01-1.03 | .003 |
| of target lesions (cm) | | | | | | |
| Prior Whipple procedure | 0.63 | 0.48-0.86 | 0.001 | 0.79 | 0.59-1.05 | .107 |
| Analgesic use | 1.13 | 0.98-1.31 | 0.087 | — | — | — |
| CA19-9 level^{b} | 1.00 | 1.00-1.00 | 0.001 | — | — | — |
| Number of metastatic sites | 1.11 | 1.03-1.20 | 0.008 | — | — | — |
| Localization of pancreatic | | | | | | |
| tumor | | | | | | |
| Body | Reference | — | 0.114 | — | — | — |
| Head | 1.06 | 0.90-1.26 | | | | |
| Tail | 1.29 | 1.07-1.56 | | | | |
| Presence of biliary stent | 0.98 | 0.81-1.18 | 0.825 | — | — | — |
| Presence of peritoneum | 1.33 | 1.04-1.71 | 0.018 | — | — | — |
| metastases | | | | | | |
| Prior chemotherapy | 0.55 | 0.37-0.81 | <0.001 | — | — | — |
| Prior radiation therapy | 0.64 | 0.43-0.95 | 0.017 | — | — | — |

Patient Factors

| | | | | | | |
|---|---|---|---|---|---|---|
| Age | 1.01 | 1.00-1.01 | 0.053 | — | — | — |
| BMI | 1.00 | 0.98-1.01 | 0.804 | — | — | — |
| Race/ethnicity | | | | | | |
| Asian | Reference | — | 0.212 | — | — | — |
| Black | 1.53 | 0.79-2.96 | | | | |
| Hispanic | 1.78 | 0.96-3.30 | | | | |
| White | 1.69 | 0.98-2.93 | | | | |
| Other | 2.54 | 1.05-6.11 | | | | |
| Sex | | | | | | |
| Female | Reference | — | 0.050 | — | — | — |
| Male | 1.15 | 1.00-1.33 | | | | |
| Weight | 1.00 | 1.00-1.01 | 0.526 | — | — | — |

^{a}The 12 demographic and clinical factors analyzed in univariable analyses but not identified as multivariable prognostic factor candidates included body surface area, height, presence of metastases in the axilla, bone, breast, groin, lung/thoracic, other, pelvis, peritoneal carcinomatosis, skin/soft tissue, and supraclavicular.
^{b}The large range of unique values demonstrated by CA19-9 (0-252,181) results in a hazard ratio and 95% confidence iinterval that are centered on 1.

Results

### Patients

Data from 861 patients (nab-paclitaxel plus gemcitabine, n=431; gemcitabine alone, n=430) enrolled in the MPACT study were included in this analysis (FIG. 7).

### Univariable and Multivariable Models

Fourteen out of a total of 32 factors examined in univariable analyses of overall survival were determined to be statistically significantly associated with survival (Table 5). These factors plus 4 others (BMI, presence of biliary stent, race/ethnicity, and weight) with known clinical relevance or proximity to the prespecified alpha-level (P<0.1) were entered into a multivariable model. Out of the 18 factors entered into the multivariable model, 6 factors remained after backward selection and were identified as being significantly associated with overall survival (Table 5).

Primary Nomogram with Internal Validation

A nomogram was generated using the 6 factors identified by multivariable analysis (FIG. 2) and was shown to predict the survival probabilities at 6, 9, and 12 months. For example, a patient with a neutrophil-to-lymphocyte ratio of 20 (25 points), a baseline albumin level of 50 g/L (14 points), a Karnofsky performance status of 100 (0 points), a sum of longest diameter of tumors of 20 cm (7 points), with liver metastasis (9 points), and that has undergone a previous Whipple procedure (0 points) has a total of 55 points, which corresponds to 6-, 9-, and 12-month predicted survival probabilities of 65%, 45%, and 31%, respectively (Table 2). For this example, the sum of the longest diameter of tumors could theoretically involve 10 liver metastases with the maximum number of 5 summed to 16 cm and the primary lesion being 4 cm for a total of 20 cm.

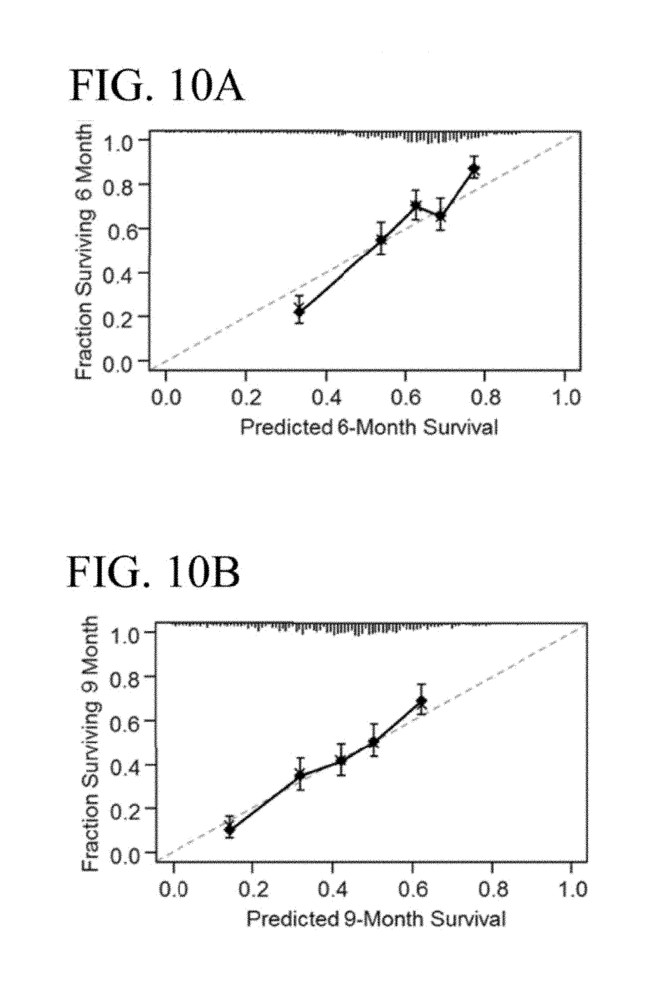

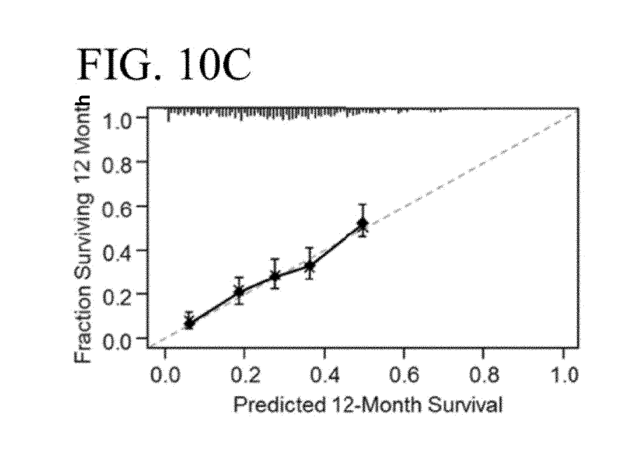

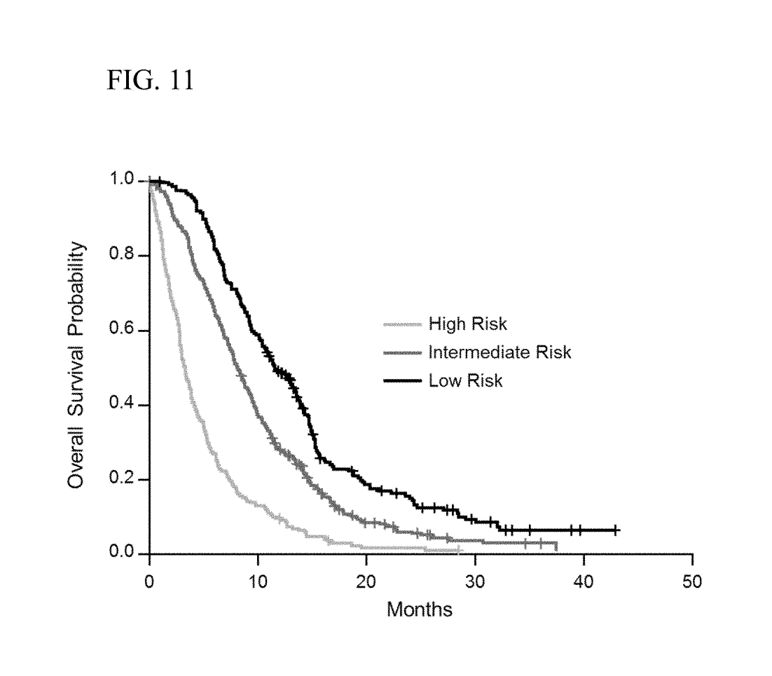

Calibration plot comparisons used to evaluate the predictive ability of the nomogram demonstrated that the mean absolute errors between the observed and predicted probabilities for 6-, 9-, and 12-month survival were 0.07, 0.03, and 0.02, respectively (FIG. 10). The nomogram was able to discriminate between low—(n=216), intermediate—(n=430), and high—(n=215) risk groups (c-index 0.67; 95% CI, 0.65-0.69) which had median overall survival values of 11.7, 8.0, and 3.3 months, respectively (FIG. 11).

Relative Contribution of Clinically Important Factors Added Individually to Primary Nomogram

In addition to the relative contribution of each factor shown in Table 2, analyses were carried out to evaluate the potential contribution of 6 clinically important factors if added individually to the primary nomogram. Age would have contributed 8 points, and number of lesions at baseline would have contributed 6 points. Presence of lung metastases, thrombosis, CA19-9 level, and number of metastatic sites each would have contributed ≤2 points (Table 6).

TABLE 6
Relative contribution of factors in a nomogram for prediction of overall survival in patients
with metastatic pancreatic cancer

| | Range per Factor | | | | | | | | |
|---|---|---|---|---|---|---|---|---|---|
| | Value | Value | | | | | | | |
| | Worth | Worth | Nomograms, Points Contributed per Factor^{a} | | | | | | |
| | Most | Least | | Primary Plus Each of the Below Factors Individually | | | | | |
| | Points | Points | | | | Number of | Number | | |
| | (Worse | (Better | | | | Metastatic | of | Lung | |
| Factor | Prognosis) | Prognosis) | Primary | CA19-9^{b} | Age | Sites | Lesions | Metastasis | Thrombosis |
| NLR | 80 | 0 | 100 | 100 | 100 | 100 | 100 | 100 | 100 |
| Albumin, g/L | 0 | 60 | 86 | 87 | 85 | 85 | 86 | 86 | 86 |
| KPS | 60 | 100 | 23 | 23 | 24 | 23 | 23 | 23 | 23 |
| SLD, cm | 50 | 0 | 18 | 18 | 20 | 19 | 16 | 18 | 18 |
| Presence of | Yes | No | 9 | 9 | 9 | 9 | 9 | 9 | 9 |
| liver metastasis | | | | | | | | | |
| Previous | No | Yes | 6 | 6 | 5 | 6 | 6 | 6 | 6 |
| Whipple | | | | | | | | | |
| CA19-9 level, | ≥400,000 | ≤100,000 | — | 2 | — | — | — | — | — |
| U/mL | | | | | | | | | |
| Age, years | 90 | 20 | — | — | 8 | — | — | — | — |
| Number of | ≤2 | ≥5 | — | — | — | 2 | — | — | — |
| metastatic sites | | | | | | | | | |
| Number of | 30 | 0 | — | — | — | — | 6 | — | — |
| lesions | | | | | | | | | |
| Lung metastasis | Yes | No | — | — | — | — | — | 1 | — |
| Thrombosis | Yes | No | — | — | — | — | — | — | 1 |

CA19-9, carbohydrate antigen 19-9;
KPS, Karnofsky performance status;
NLR, neutrophil-to-lymphocyte ratio;
OS, overall survival;
SLD, sum of longest tumor diameters.
^{a}Points contributed to the nomogram as a measure of the relative importance of each factor; the greater the number, the greater the factor's contribution to the model.
^{b}The CA19-9 nomogram was created using data from a smaller subset of patients (n = 634) because non-secretors (non-expressors) were excluded.

Discussion

This prognostic nomogram demonstrated that survival could be more accurately estimated using baseline factors, including neutrophil-to-lymphocyte ratio, albumin level, Karnofsky performance status, sum of the longest diameter of target lesions, presence of liver metastases, and previous Whipple procedure. This nomogram may allow physicians and patients to make more informed and individualized decisions about systemic treatment and management of metastatic pancreatic cancer.

The current analysis identified CA19-9 level as a potential predictive factor at the univariable level; however, the factor did not ultimately remain significant in the final multivariable model. When CA19-9 was forced into the primary nomogram, it only provided a minimal additional contribution

Current prognostic markers of disease are generally qualitative with little ability to account for the impact of a given factor in context of the overall patient profile. These findings indicate that certain factors may be more influential in estimating a patient's prognosis than others, and the nomogram presented herein may allow more accurate and individualized risk prediction by differentially weighting the factors within. The analysis of relative contribution for each factor indicated that the largest contributors to survival prognosis were neutrophil-to-lymphocyte ratio and albumin level. Furthermore, internal validation of the nomogram demonstrated that it was reliable for the prediction of survival in low-, intermediate-, and high-risk groups, as indicated by the c-index score of 0.67. This indicates that it should be possible to establish risk categorization in metastatic pancreatic cancer that might be applied to future trial stratification.

The factors presented in this nomogram are simple to evaluate from routinely collected information at baseline. Although the sum of longest diameter of target lesions and neutrophil-to-lymphocyte ratio may be less familiar to some physicians, both should be readily obtainable at little additional costs using existing patient measurements. Neutrophil and lymphocyte counts are routinely measured before treatment, and physicians can use a simple algorithm to calculate neutrophil-to-lymphocyte ratio (Please see Supplementary Material). The sum of longest diameters of target lesions could also be obtained from radiographic scans. The remaining 4 factors (albumin level, Karnofsky performance status, presence of liver metastasis, and whether a patient has undergone a previous Whipple procedure) are all routinely collected in clinical practice.

### Conclusions

The present nomogram can be used to predict the survival of individual patients with metastatic pancreatic cancer treated with chemotherapy nab-paclitaxel plus gemcitabine or gemcitabine alone. A more accurate estimation of survival may guide physicians and patients in their management decisions regarding metastatic pancreatic cancer (i.e., standard treatment, no treatment, or experimental treatment). Future clinical trials may also consider nomograms to guide patient stratification.

1. A nomogram for determining a survival probability of an individual having metastatic pancreatic cancer, the nomogram comprising:
one or more factor scales comprising values for one or more factors;
a points scale comprising points values;
a total points scale comprising total points values; and
a prediction scale;
wherein the one or more factor scales are correlated with the points scale and wherein the total points scale is correlated with the prediction scale,
wherein in response to receiving values for the one or more factors, correlating the values for the one or more factors with the points scale to determine one or more points values, combining the one or more points values to determine a total points value, correlating the total points value with the prediction scale, and outputting a survival probability based on the prediction scale.

2-15. (canceled)

16. The nomogram of claim 1, wherein the one or more factors comprise neutrophil to lymphocyte ratio, albumin level, Karnofsky performance status, sum of longest diameter of target lesions, liver metastasis, or previous Whipple procedure.

17. The nomogram of claim 1, wherein the one or more factors comprise CA 19-9 level.

18. The nomogram of claim 1, wherein the one or more factors comprise age.

19. The nomogram of claim 1, wherein the one or more factors comprise number of metastatic sites.

20. The nomogram of claim 1, wherein the one or more factors comprise number of lesions.

21. The nomogram of claim 1, wherein the one or more factors comprise presence of lung metastasis.

22-23. (canceled)

24. The nomogram of claim 1, wherein the individual has received treatment with gemcitabine.

25. The nomogram of claim 1, wherein the individual has received treatment with a nanoparticle composition comprising paclitaxel and albumin.

26. The nomogram of claim 1, wherein the survival probability is calculated at 6 months.

27-28. (canceled)

29. The nomogram of claim 1, wherein the survival probability is outputted as a range of time before the individual is likely to die.

30. A method of using the nomogram of claim 1, comprising determining one or more factors and providing a survival probability.

31. A method to predict a survival probability of an individual diagnosed with metastatic pancreatic cancer comprising
receiving values for one or more factors for an individual;
determining a separate points value for each of the one or more factors based upon one or more factor scales that are correlated with a points scale;
combining each of the separate point values together to yield a total points value; and
correlating the total points value with a prediction scale to predict the survival probability of the individual.

32. A computer-implemented method to predict a survival probability of an individual diagnosed with metastatic pancreatic cancer comprising:
receiving one or more input values for one or more factors, wherein the one or more input values are associated with the individual;
after receiving the one or more input values, determining, for each of the one or more factors, a respective points value based upon a points scale and a respective factor scale correlated with the points scale;
aggregating the respective point values for the one or more factors to yield a total points value;
correlating the total points value with a prediction scale to predict the survival probability of the individual; and
providing one or more outputs based on the predicted survival probability of the individual.

33-61. (canceled)

62. A method of treatment comprising using the nomogram of claim 1 to calculate a survival probability and providing a treatment recommendation to the individual.

63. The method of claim 62, further comprising treating the individual.

64-67. (canceled)

68. A method of patient stratification comprising calculating a survival probability using the nomogram of claim 1.
